(19) United States
(12) Patent Application Publication
(10) Pub. No.: US 2025/0352158 A1
(43) Pub. Date: Nov. 20, 2025

(54) DIASTOLIC FUNCTION EVALUATION SYSTEM AND ASSOCIATED METHODS

(71) Applicant: ADVENTIST HEALTH SYSTEM/SUNBELT, INC, Altamonte Springs, FL (US)

(72) Inventors: Richard J. Moro, Winter Park, FL (US); Kapil Kapoor, Orlando, FL (US)

(21) Appl. No.: 18/873,192

(22) PCT Filed: Sep. 25, 2023
(86) PCT No.: PCT/US2023/033611

§ 371 Date: Dec. 9, 2024

Related U.S. Application Data
(60) Provisional application No. 63/409,436, filed on Sep. 23, 2022.
(60) Provisional application No. 63/420,267, filed on Oct. 28, 2022.

(51) Int. Cl. A61B 6/50; A61B 5/026; A61B 6/00; A61B 8/00; A61B 8/06; G06T 7/00; G16H 30/40; G16H 80/00

(52) U.S. Cl. CPC A61B 6/503; A61B 5/0263; A61B 6/507; A61B 6/5217; A61B 8/065; A61B 8/5223; G06T 7/0012; G16H 30/40; G16H 80/00; G06T 2207/10081; G06T 2207/10088; G06T 2207/30048; G06T 2207/30104

(57) ABSTRACT

Methods, systems, and devices for evaluating function of a heart are provided. The methods can include use of one or more imaging modalities to assess diastolic function. The methods, systems, and devices can employ various features of a cardiac volume curve to assess cardiac function. The methods, systems, and devices can be used to guide diagnosis, therapy, and/or training for sick or well patients. Methods, systems, and devices for assessing likelihood of hospital readmission for a heart failure patient are also provided.

### RELATED APPLICATIONS

This application claims priority under 35 U.S.C. § 119 to U.S. Provisional Application No. 63/409,436 filed Sep. 23, 2022 and U.S. Provisional Application No. 63/420,267, filed Oct. 28, 2022, the entire content of each of which is incorporated by reference herein.

### TECHNICAL FIELD

The present disclosure relates to evaluation of heart function, including use of various imaging modalities to assess diastolic heart function. The methods and devices disclosed herein may be used to assess heart health, improve treatments, and/or optimize cardiac performance for sick or healthy subjects.

### BACKGROUND

Assessment of cardiac function can include analysis of both systolic and diastolic function. Many believe that systolic function assessment is easy, and diastolic function assessment is hard—a statement that has been reiterated in many diastolic-focused papers. Systolic function has complexities associated with timely myocardial stimulation and viability to name a few. It is most often assessed using ejection fraction, a single number, but stroke volume (SV) and the rates of contraction and relaxation can also be easily measured. Assessment of diastolic function is more complicated.

Dynamic systolic changes cannot occur, however, without adequate filling volume, which occurs during diastole. Currently, appreciating influences on left ventricular filling requires studying over a dozen echo/Doppler measurements, and these complexities often leading to lack of consensus and confusion. The American Society of Echocardiography (ASE) 2016 Guidelines (hereinafter “ASE Guidelines”) have significantly simplified diastolic performance grading into Normal, Grades 1, 2, 3, and Indeterminate, but these grades focus on velocity relationships primarily related to a variable orifice, the mitral valve, which, because of its attachment points to the left atrium and left ventricle, lends itself to unpredictable mitral valve orifice deformation influences. These deformations often cause increases in velocity suggesting increased volume delivery but may be the result of decreasing orifice parameters resulting in decreased volume exchange.

Diastolic performance is dependent on volume transfer from the left atrium (LA) to the left ventricle (LV). How this LA-to-LV volume transfer occurs is not as important during sedentary or low-activity periods. It is sufficient that the required volume is delivered prior to ventricular systole. However, with increasing output demands or worsening pathologies, a better understanding of how and when the LA-to-LV transfer occurs becomes more important. But current methods for assessing diastolic function focus on velocity measurements, and such measures may be insufficient in the presence of various conditions.

Volume Curve (VC) analysis is a new method based on an old theme, namely, using left ventricular volumes and filling rates rather than echo/Doppler velocities to study and classify diastolic performance. The ventricular VC can be generated utilizing multiple cardiac imaging techniques. It represents the culmination or sum of all factors affecting left ventricle (LV) volume transfer. Providing an enclosed pump with hydraulic valving controlling ingress and egress, the LV VC offers an excellent model for diastolic evaluation. From right atrium (RA) volume return to systemic pressure, each component plays a role affecting left atrial and LV preload, filling, and afterload.

When analyzing diastolic function, a volume curve provides delivery rates, via slopes, and volume delivery during three separate components of the curve. It is these relationships that more exactly define pressure and compliance relationships between the LA and LV subsequently affecting total ventricular volume.

Volume characterization of diastole provides a unique assessment of the sedentary heart that may prove predictive of exertional response. Accordingly, the present disclosure provides improved methods for cardiac assessment including use of volume measurements to assess diastolic performance.

### SUMMARY

According to various embodiments, a method for assessing cardiac function is provided. In some embodiments, the method includes receiving or accessing cardiac data obtained from imaging one or more complete cardiac cycles of the heart. The method also includes identifying a diastolic phase of the heart from the cardiac data and identifying in the diastolic phase an early filling phase, an intermediate filling phase, and an atrial contraction phase from the cardiac data. The method also includes determining a left ventricular filling rate (R1) during the early filling phase from the cardiac data; determining a left ventricular filling rate (R2) during the intermediate filling phase from the cardiac data; determining a first left ventricular filling volume (E) during the early filling phase from the cardiac data; and determining a second left ventricular filling volume (A) during the atrial contraction phase from the cardiac data. The method also includes generating a diastolic function classification based on at least R1, R2, E, and A.

In some embodiments, the method also includes determining a filling volume of the heart. In some embodiments, the diastolic function classification is based on at least a ratio of R1:R2.

In some embodiments, the classification based on E is based on at least one of E as a percentage of a ventricular total filling volume (E %), as a percentage of a body surface area (Ei), or a combination of the two (Ei %). In some embodiments, the classification based on E is based on E as a percentage of the filling volume (E %).

In some embodiments, the classification based on A is based on at least one of A as a percentage of the ventricular total filling volume (A %), as a percentage of a body surface area (Ai), or a combination of the two (Ai %). In some embodiments, the classification based on A is based on A as a percentage of filling volume (A %).

In some embodiments, the classification is further based on a minute ventricular output (MVO) that is body surface area (BSA) indexed (MVOi). In some embodiments, the method further also includes calculating a left ventricular filling rate (R3) during an atrial contraction phase, wherein A is determined based on R3.

In some embodiments, the diastolic function classification includes one of normal, an early enhanced, a mid-enhanced, an atrial enhanced, an aerobic enhanced, a delayed filling, a restrictive/constrictive or a hypovolemic classification. In some embodiments, the diastolic function classification includes four classifications selected from the group consisting of: normal, an early enhanced, a mid-enhanced, an atrial enhanced, an aerobic enhanced, a delayed filling, a restrictive/constrictive, and a hypovolemic classification.

In some embodiments, the normal classification corresponds to an R1:R2 ratio of greater than about 4 and less than about 16, an E % of greater than about 50% and less than about 70%, and an A % of greater than about 25%.

In some embodiments, the early enhanced classification corresponds to an R1:R2 ratio of greater than about 4 and less than about 16, an E % of greater than about 50% and less than about 70%, and an A % of less than about 25%; or the early enhanced classification corresponds to an R1:R2 ratio of less than about 16, and an E % of greater than about 70%; or both.

In some embodiments, the mid-enhanced classification corresponds to an R1:R2 ratio of less than about 16, an E % of less than about 50%, and an A % of less than about 40%.

In some embodiments, the atrial enhanced classification corresponds to an R1:R2 ratio of less than about 16 and greater than about 4, an E % of less than about 50%, and an A % of greater than about 30%; or the atrial enhanced classification corresponds to an R1:R2 ratio of greater than about 16, an A % of greater than about 30% and a minute ventricular output indexed by body surface area (MVOi) of less than about 2200; or both.

In some embodiments, the aerobic enhanced classification corresponds to an R1:R2 ratio of greater than about 16, an A % of greater than about 30%, and a minute ventricular output indexed by body surface area (MVOi) of greater than about 2200.

In some embodiments, wherein the delayed filling classification corresponds to an R1:R2 ratio of less than about 4, and an E % of greater than about 50% and less than about 70%.

In some embodiments, the restrictive/constrictive classification corresponds to an R1:R2 ratio of greater than about 16, and an A % of less than about 30%.

In some embodiments, the hypovolemic classification corresponds to an R1:R2 ratio of less than about 4, an E % of less than about 50% and an A % of greater than about 30%.

In some embodiments, the method also includes displaying information regarding the generated diastolic function classification or a representation of the generated diastolic function classification on a display. In some embodiments, the method also includes storing information regarding the generated diastolic function classification. In some embodiments, the method also includes transmitting information regarding the generated diastolic function classification to a health care provider or health care system of the subject. In some embodiments, the method also includes transmitting an alert to a health care provider or a health care system of the subject where the generated diastolic function classification is hypovolemic classification.

In some embodiments, the method also includes imaging the heart during one or more complete cardiac cycles to generate the cardiac data. In some embodiments, imaging the heart includes imaging using at least one of echocardiography, computed tomography (CT), and magnetic resonance imaging.

Also provided is a system for automatic evaluation of cardiac function. In some embodiments, the system includes memory and at least one processor, and a diastolic function classification module comprising instruction. The instructions are enabled upon execution in the memory of the host computing platform to: receive or access cardiac data obtained from imaging one or more complete cardiac cycles of a heart; identify a diastolic phase and a systolic phase of the heart based on the cardiac data; identify in the diastolic phase an early filling phase, an intermediate filling phase, and an atrial contraction phase; determine a left ventricular filling rate (R1) during the early filling phase from the cardiac data; determine a left ventricular filling rate (R2) during the intermediate filling phase from the cardiac data; determine a ventricular filling volume (E) during the early filling phase from the cardiac data; determine a left ventricular filling volume (P) prior to the atrial contraction phase from the cardiac data; determine a ventricular total filling volume (FV) from the cardiac data; determine a left ventricular filling volume (A) during the atrial contraction phase from the cardiac data; and generate a diastolic function classification based on R1, R2, E and A.

In some embodiments, the system also includes a display, and the instructions further include instructions to display information regarding the generated diastolic function classification or a representation of the generated diastolic function classification on the display.

In some embodiments, the instructions also include instructions to store information regarding the generated diastolic function classification.

In some embodiments, the instructions also include instructions to transmit information regarding the generated diastolic function classification to a health care provider or health care system of the subject. In some embodiments, the instructions also include instructions to transmit an alert to a health care provider or a health care system of the subject where the generated diastolic function classification is hypovolemic classification.

In some embodiments, the system also includes imaging instrumentation for imaging the heart. In some embodiments, the imaging instrumentation is configured for at least one of echocardiography, computed tomography (CT), and magnetic resonance imaging.

In some embodiments, a system for automatic evaluation of cardiac function is provided. The system can comprise a host computing platform comprising computing devices each with memory and at least one processor, and a diastolic function classification module comprising computer program instructions enabled upon execution in the memory of the host computing platform to perform one or more of the aforementioned methods.

In some embodiments, a non-transitory computer readable medium is provided. The medium can store instructions that when executed by one or more processors of a device or system, cause the device or system to perform one or more of the aforementioned methods.

In some embodiments, a computer program product for assessing cardiac function of a heart is provided, the computer product can include a non-transitory computer readable storage medium having program instructions embodied therewith, the program instructions executable by one or more processors of a device or system to cause the device or system to receive or access cardiac data obtained from imaging one or more complete cardiac cycles of a heart; identify a diastolic phase and a systolic phase of the heart based on the cardiac data; identify in the diastolic phase an early filling phase, an intermediate filling phase, and an atrial contraction phase; determine a left ventricular filling rate (R1) during the early filling phase from the cardiac data; determine a left ventricular filling rate (R2) during the intermediate filling phase from the cardiac data; determine a ventricular filling volume (E) during the early filling phase from the cardiac data; determine a left ventricular filling volume (P) prior to the atrial contraction phase from the cardiac data; determine a ventricular total filling volume (FV) from the cardiac data; determine a left ventricular filling volume (A) during the atrial contraction phase from the cardiac data; and generate a diastolic function classification based on R1, R2, E and A.

In some embodiments, method for identifying a heart failure patient having an increased likelihood of hospital readmission is provided. The method can comprise performing one or more of the aforementioned methods to obtain a diastolic function classification of the patient's heart; determining whether the patient falls in a normal group having a first likelihood of hospital readmission or in an abnormal group having a second likelihood of hospital readmission higher than the first likelihood based on the diastolic function classification; and where the patient falls in the abnormal group, identifying the patient as having an increased likelihood of hospital readmission within a time period.

In some embodiments, the diastolic function classifications include normal, atrial enhanced, early enhanced, mid enhanced, delayed filling, restrictive/constrictive, and hypovolemic, wherein the normal group includes the diastolic function classifications normal, atrial enhanced, and early enhanced, and wherein the abnormal group includes the diastolic function classifications mid enhanced, delayed filling, restrictive/constrictive, and hypovolemic.

In some embodiments, the method further includes determining a Volume Curve Moro Index (VCMI) from the cardiac data, the VCMI corresponding to a likelihood of hospital readmission within the diastolic function classification, with a lower VCMI value corresponding to a higher likelihood of hospital readmission within the specified time period and a higher lower VCMI value corresponding to a lower likelihood of hospital readmission within the specified time period.

In some embodiments, the time period falls within a range of 2 days to 100 days.

In some embodiments, the time period falls within a range of 10 days to 100 days

In some embodiments, the time period falls within a range of 14 days to 100 days.

In some embodiments, the method further comprises transmitting an alert or a communication where the patient is identified as having an increased likelihood of hospital readmission; or displaying an alert where the patient is identified as having an increased likelihood of hospital readmission.

In some embodiments, system for identifying a heart failure patient having an increased likelihood of hospital readmission is provided. The system can comprise a memory and at least one processor; and a diastolic function classification module comprising instructions enabled upon execution in the memory to perform one or more of the aforementioned methods.

### BRIEF DESCRIPTION OF DRAWINGS

Embodiments of the invention are described by way of example with reference to the accompanying drawings in which:

FIG. 1A schematically depicts a method for assessing cardiac function of a heart of a subject in accordance with some embodiments.

FIG. 1B is a flowchart illustrating a decision tree for generating a diastolic function classification from cardiac data (e.g., for assigning a VC class) in accordance with some embodiments.

FIG. 2 is a Table illustrating classification features for the disclosed cardiac function assessment method in accordance with some embodiments.

FIG. 3A illustrates an exemplary prior art volume-time curve.

FIG. 3B illustrates an exemplary prior art first derivative (dV/dT) of the volume-time curve of FIG. 3A.

FIG. 3C illustrates another exemplary time prior art time volume curve.

FIG. 3D illustrates an exemplary prior art first derivative (dV/dT) of the volume-time curve of FIG. 3C.

FIG. 4A is an illustrative graph of a volume curve overlaid with left ventricular inflow tract (LVIT) Doppler aligned based on ECG.

FIG. 4B is a photo of the volume curve overlaid with the left ventricular inflow tract (LVIT) Doppler aligned based on ECG

FIG. 5 is an exemplary cardiac volume-time curve aligned with an exemplary ECG and showing systole and diastole.

FIG. 6 is an exemplary cardiac volume-time curve with shading to illustrate the early filling, mid-filling, and atrial contribution to filling during diastole.

FIG. 7 is an exemplary cardiac volume-time curve with shading to illustrate late filling during diastole.

FIG. 8 is an exemplary cardiac volume-time curve illustrating calculation of filling rate (R1) during an early filling phase.

FIG. 9 is an exemplary cardiac volume-time curve illustrating calculation of filling rate (R2) during a mid-filling phase.

FIG. 10 is an exemplary cardiac volume-time curve illustrating calculation of R1, R2, early filling volume (E), and filling volume (FV).

FIGS. 11A and 11B are an illustrative graph and a photo of a VC with an enhanced early filling classification.

FIGS. 12A and 12B are an illustrative graph and a photo of a VC with a delayed filling classification.

FIGS. 13A and 13B are an illustrative graph and a photo of a VC with a restrictive/constrictive filling classification.

FIGS. 14A and 14B are an illustrative graph and a photo of a VC with a mid-filling enhanced classification.

FIG. 15 is a graph illustrating various measurements and calculations relating to an exemplary volume curve.

FIG. 16 is a graph illustrating various measurements and calculations relating to a complete diastolic-systolic volume curve capture.

FIG. 17 schematically depicts an embodiment of a system for performing cardiac function assessment.

FIG. 18 schematically depicts hardware and software components capable of being utilized to implement the system of the present disclosure.

FIG. 19 schematically depicts a system that both obtains images of a heart and performs diastolic function classification in accordance with some embodiments.

FIG. 20 provides Kaplan-Meier Curves of admissions following the INDEX echo for 30 and 90 days as estimated using VC classification to group into Normal and Abnormal groups and as estimated using the ASE grades to group into a lower grade abnormality group and into a higher grade abnormality group.

### DETAILED DESCRIPTION OF EMBODIMENTS

A description of embodiments of the invention will now be described more fully hereinafter with reference to the accompanying drawings, in which the embodiments are shown by way of illustration and example. This invention may, however, be embodied in many forms and should not be construed as limited to the embodiments set forth herein. Rather, these embodiments are provided so that this disclosure will be thorough and complete, and will fully convey the scope of the invention to those skilled in the art. Like numerals refer to like elements.

Some embodiments of the present invention relate to assessment of cardiac function. Specifically, embodiments relate to assessment of cardiac diastolic function using various aspects of the cardiac volume curve. These aspects of the VC can include the rates and/or volumes of filling during early or mid-filing phases as well as filling volumes at various stages of diastole. Compared to current classification systems, the present methods and systems can allow better classification of diastolic function. This improved classification can help guide treatment or predict clinical outcomes.

The ventricular volume curve (VC) can be generated using various cardiac imaging techniques, which are discussed further below. The volume curve represents the combined influence of various aspects of physiology and pathology affecting diastolic function (e.g., RA return volume or systemic pressure). Because of this combined effect, the LV VC offers an excellent model for diastolic evaluation, while also providing other information about cardiac function such as filling volume (FV), stroke volume (SV) and/or cardiac output (CO).

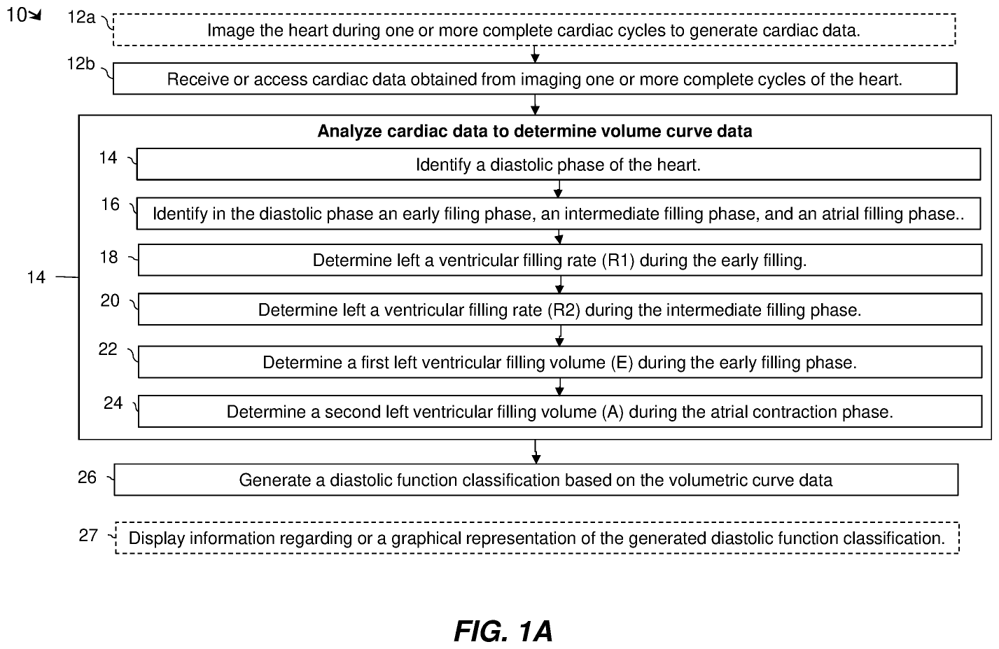

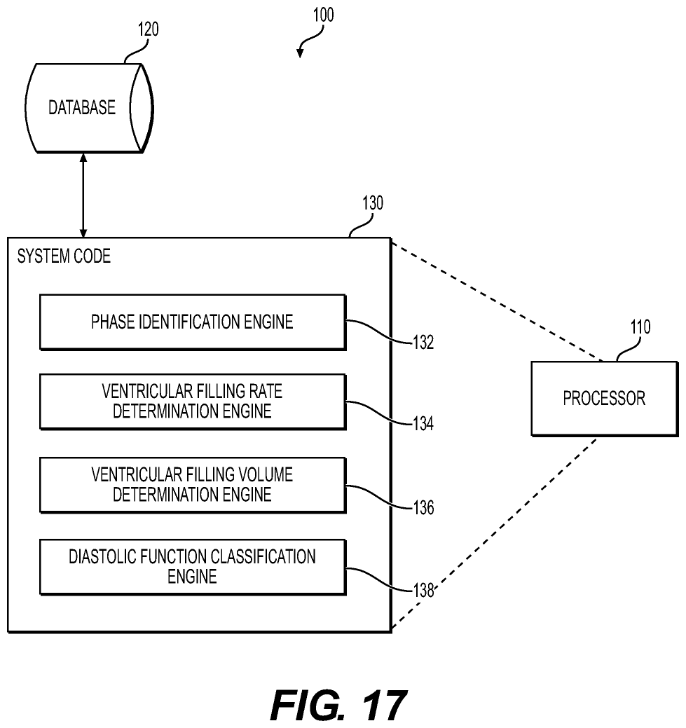

A method 10 for assessing cardiac function of a heart of a subject is schematically depicted in FIG. 1A. The cardiac function assessment can be conducted by a system or device including one or more processors (e.g., a computing system, a computing device, an imaging system, an ultrasound system, etc.) based on data obtained from imaging one or more complete cardiac cycles of the heart (i.e., cardiac data). In some embodiments, the same system or device that performs the cardiac imaging to generate the cardiac data may also perform the cardiac function assessment. In some embodiments, the cardiac function assessment may be performed by a different system or device that a system or device that performs the cardiac imaging to generate the cardiac data. In some embodiments, a portion of the cardiac function assessment is performed by a system or device that generates performs the cardiac imaging to generate the cardiac data and another portion of the cardiac function assessment is performed by a different system or device. Further description of devices and systems for implementing embodiments of the invention appears below with respect to FIGS. 17 to 19.

In some embodiments, the method includes imaging the heart during one or more complete cardiac cycles for generating the cardiac data (12a). In some embodiments, the imaging is via echocardiography (e.g., 3D echocardiogram data). Other cardiac imaging techniques that may be employed to generate the cardiac data, include, but are not limited to cardiovascular magnetic resonance (CMR), cardiac computer tomography (CCT), cardiac nuclear medicine (CNM), and electron beam tomography (EBT). In some embodiments, a system used to obtain images to generate the cardiac data is or includes a 3D ultrasound imaging system. In some embodiments, the method includes receiving or accessing cardiac data from imaging one or more complete cycles of the heart (12b). In some embodiments, the method does not include imaging the heart, but instead the received or accessed cardiac data was previously obtained. The dotted line around 12a indicates that it may not be included in some embodiments.

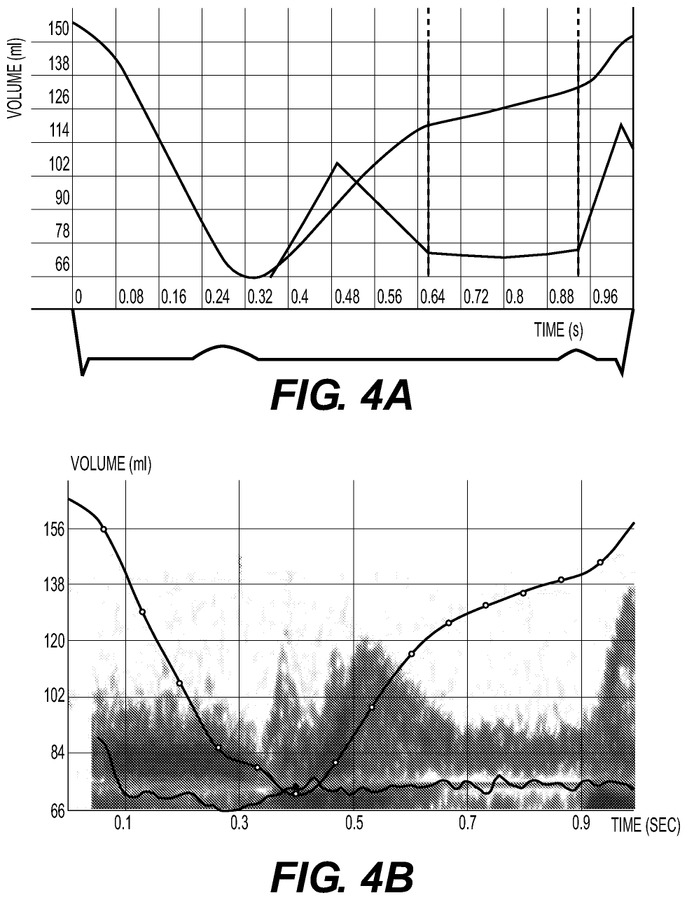

The method includes analyzing the cardiac data (e.g., 3D image data) to determine volume curve data (13). In some embodiments, this analysis to determine the volume curve data includes identifying a diastolic phase of the heart from the cardiac data (14). In some embodiments, the analysis also includes identifying an early filling phase, and intermediate filling phase and an atrial contraction phase in the diastolic phase from the cardiac data (16). In some embodiments, the analysis includes determining a left ventricular filling rate (R1) during the early filling phase from the cardiac data (18). In some embodiments, the analysis includes determining a left ventricular filling rate (R2) during the intermediate filling phase from the cardiac data (20). In some embodiments, the analysis includes determining a first left ventricular filling volume (E) during the early filling phase from the cardiac data (22) and determining a second left ventricular filling volume (A) during the atrial contraction phase from the cardiac data (24). Further explanation of how to determine R1, R2, E, and A is provided below with respect to FIGS. 5 to 13B. The method also includes generating a diastolic function classification based on the volumetric curve data (26) (e.g., based on at least R1, R2, E, and A), which is described herein with respect to FIG. 1B. In some embodiments, the method also includes displaying information regarding or a graphical representation of the generated diastolic function classification using a display of the system or device (27). In some embodiments, the method also includes storing information regarding the generated diastolic function information. In some embodiments, the method also includes transmitting information regarding the generated diastolic function classification to a health care provider or health care system of the subject. In some embodiments, the method also includes transmitting a notification or an alert to a health care provider or a health care system of the subject where the generated diastolic function classification is Hypovolemic Classification.

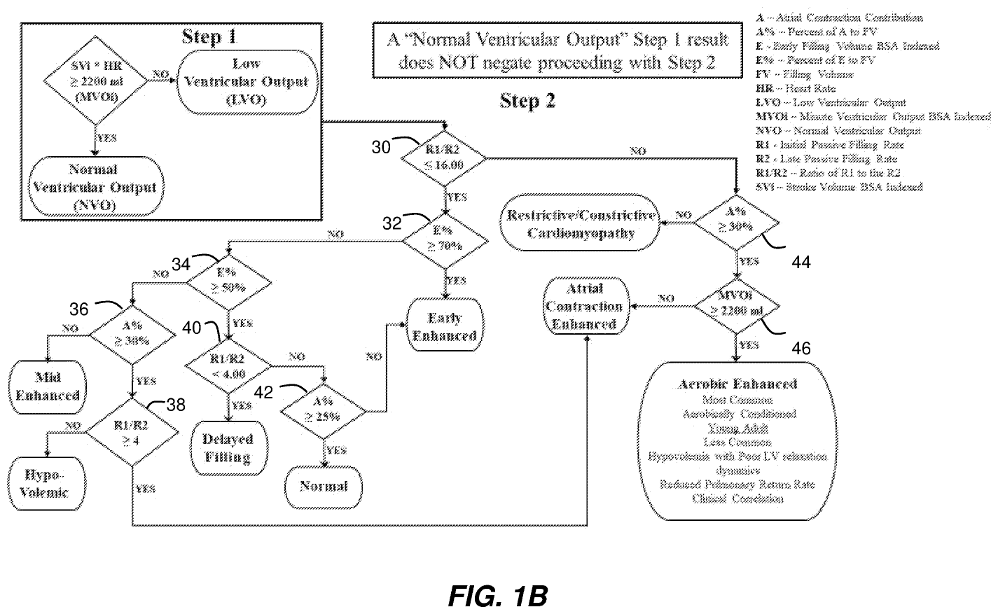

FIG. 1B is a flow chart illustrating steps in generating a diastolic function classification based on at least R1, R2, E and A (26 of FIG. 1A) according to some embodiments. FIG. 2 is a Table illustrating classification features for the disclosed cardiac function assessment method according to some embodiments.

As shown at Step 1 of FIG. 1B, the method can include first determining if ventricular output is normal from the cardiac data. Generally, normal ventricular output has been indexed using body surface area and a stroke volume of greater than 2200 ml. In some embodiments, the cutoff for normal ventricular output may be set to a different level (e.g., 1800 ml). The specific Body Surface Area (BSA)-indexed SV may vary based on the type of imaging modality and patient population under consideration. Therefore, regardless of diastolic function classification, a patient may have normal or low cardiac output. As explained further below, the present methods and systems may provide a classification based on systolic function or cardiac output, and further provide a classification based on diastolic function.

The diastolic evaluation begins at Step 2. It should be noted, however, that the diastolic classification can be performed without performing Step 1, or when Step 1 was performed independently or separately. An analysis of a volume curve (VC) is performed for evaluation of diastolic function to determine various values required for the evaluations in Step 2 of FIG. 1B. The specific sections and elements of the volume curve analysis are described below, but generally, the method includes identifying a diastolic phase of a heart based on the cardiac data; identifying in the diastolic phase an early filling phase, an intermediate filling phase, and an atrial contraction phase; determining a left ventricular filling rate (R1) during the early filling phase; determining a left ventricular filling rate (R2) during the intermediate filling phase; determining a first left ventricular filling volume (E) during the early phase; determining a second left ventricular filling volume corresponding to an atrial contraction phase (A); and providing a diastolic function classification based on R1, R2, E, and A. As explained below and illustrated in the flowchart of FIG. 1 and the Table of FIG. 2, the diastolic classification may include one, some, or all of normal, an early enhanced, a mid-enhanced, an atrial enhanced, an aerobic enhanced, a delayed filling, a hypovolemic, and a restrictive/constrictive classification in accordance with some embodiments. The following sections describe calculation of each of these and other relevant cardiac volumes and rates.

Referring more specifically to FIG. 1B, the method includes determination of LV filling rates (R1 and R2) during early and intermediate filling stages. Next, a ratio of R1:R2 is determined, and the R1:R2 (hereinafter referred to as “R1:R2”, “the Ratio”, “R1:R2 Ratio”, or “R1/R2”)) is evaluated. This ratio is used at multiple different decision points in the method (e.g., at decision points 30, 38, and 40 of FIG. 1B). In addition, the E and A are calculated based on the volume curve data. Values of E and A are also used at multiple different decision points in the method (e.g., E at decision points 32 and 34, and A at decision points 36, 42, and 44 of FIG. 1B). The ventricular volume (P) at the termination of the passive filling phase which when subtracted from the Filling Volume (FV) provides the atrial contraction phase filling volume (A) (A=FV−P). Further explanation of the determination of E and A, and modified indicators of E and A based on cardiac data are provided herein.

It should be understood that although methods and processes include a determination of E and/or A for providing the diastolic classification, in some embodiments, E and A are refined to produce normalized values and/or indexed values for diastolic classification. For example, in some embodiments, E and A may be normalized to Filling Volume (FV) and represented as a percentage of FV, e.g., A %=A/FV and E %=(E/FV), or represented as a percentage of Stroke Volume (SV) for use in the diastolic classification. In some embodiments, indexes of the E and A measurements may be determined where E and A are indexed to Body Surface Area (BSA), i.e., Ai %=(A/FV/BSA) and Ei %=(E/FV/BSA), to Body Mass Index (BMI), i.e., Aj %=(A/FV/BMI) and Ej %=(E/FV/BMI), or to some other measurement and the indexed values used in classification. These further refined measurements can be derived from E and A and employed in the methods and classification processes described herein. Although the process shown in FIG. 1B and FIG. 2 (Table) includes classification based on A and E normalized to Filling Volume (A % and Ei %) for illustrative purposes, a similar classification can be made using A and E values that are normalized to SV, indexed to BSA, or indexed to BMI to provide analogous results.

Using the different factors and information derived from a volume curve, diastolic function may be classified in one of multiple different categories (e.g., 5 or more categories, 6 or more categories, 7 of more categories, 8 or more categories). In the embodiment corresponding to the table in in FIG. 2, the classification includes 8 different categories with two of those categories having multiple different types. These categories/classifications can include some or all of (1) Normal, (2) Early Enhanced filling (which may have one of two types), (3) Mid-enhanced filling, (4) Atrial Enhanced filling (which may have one of two types), (5) Aerobic Enhanced filling, (6) Delayed filling, (7) Restrictive/Constrictive, and (8) Hypovolemic. The decision points in the decision tree of FIG. 1B and the classification Table of FIG. 2 provide parameters for each possible classification.

One of ordinary skill in the art, in view of the present disclosure, will appreciate that embodiments for cardiac function evaluation may employ a flowchart having a different structure that than of FIG. 1, may employ a different classification equation than that presented above, and may employ different cutoffs for various criteria than those employed in the flow chart of FIG. 1 and the table of FIG. 2 and still fall within the scope of the present invention.

The evaluation of the VC and classifications derived therefrom are based on a collection of data from 111 subjects at AdventHealth (Florida Hospital), Orlando, Florida. The data was collected from a database used to study and program bi-ventricular pacemakers. A baseline and final echocardiogram including a 3D full-volume capture of the left ventricle was captured for each subject. QLab, a software program included on the PHILLIPS® echo imager, was used to generate the VC. Although the data and classification were collected using echocardiogram with 3D capture, it should be appreciated that any other imaging modality that provides data relating to LV volume versus time during the cardiac cycle can be used in accordance with the present invention. Further, although the data relating to cardiac function is referred to as the “volume curve” or “VC,” throughout, it should be appreciated that the data need not be converted into a “curve” or “graph.” The underlying data relating to LV volume versus time during a cardiac cycle may be analyzed without producing a graph or “curve,” but such curves may be produced using imaging and software systems described herein if such visualization will aid in treatment or diagnosis.

In order to explain the terminology and method in more detail, the following sections provide some information on basics of cardiac volume curves and volume curve calculations and measurement, followed by an explanation of the classification method and use with imaging, diagnostic, or software systems.

### Volume Curve Basics

Left ventricular volume curves provide a visual presentation of the changes occurring throughout the cardiac cycle. For comparison, the accepted format illustrated in the literature is used to demonstrate VC features. However, as explained above, the present methods need not use a graph or actual “curve” but may rely on underlying data reflecting the LV volume changes over time throughout a cardiac cycle.

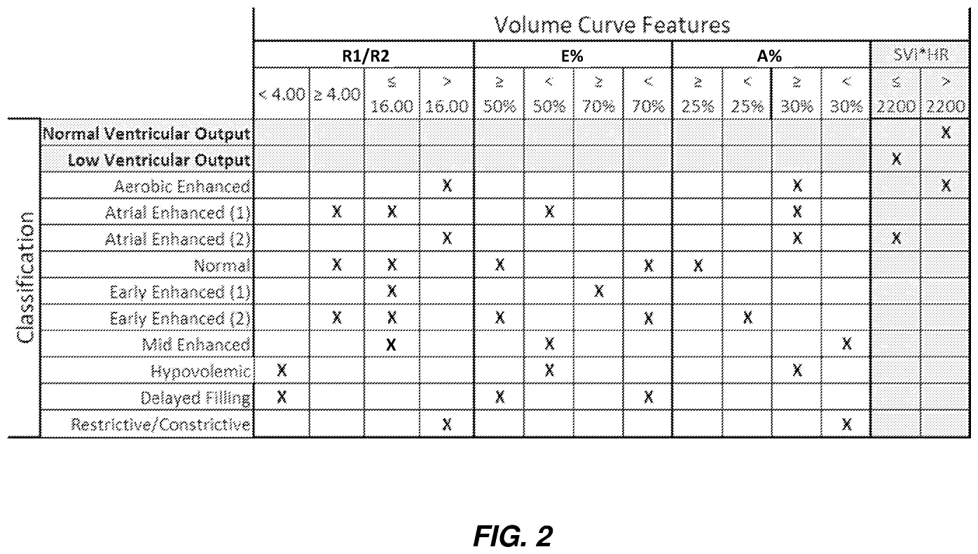

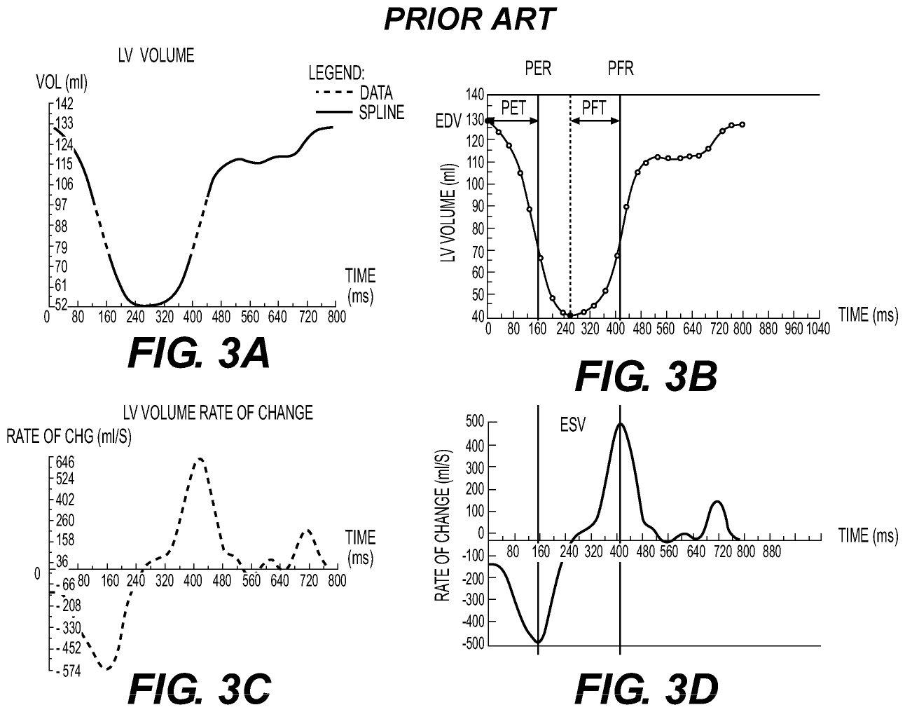

FIGS. 3A-3D illustrate exemplary volume-time curves (FIGS. 3A and 3B) and first derivatives of volume-time (dV/dT) (FIGS. 3C and 3D). (From Yan et al. “Impact of type 2 diabetes mellitus on left ventricular diastolic function in patients with essential hypertension: evaluation by volume-time curve of cardiac magnetic resonance,” Cardiovasc Diabetol, 2021 Mar. 25; 20(1):73. In FIGS. 3A-3D, the x-axis is time in seconds with the range initiating at “0” timed to the peak of the ECG “R” wave with evenly divided intervals terminating with the following “R” wave. The y-axis is volume in milliliters with the numeric series equally divided. The following example VCs (FIGS. 4-14) show the end systolic LV volume (labeled ESV in FIG. 3C, 40 ml), and the end diastolic volume (EDV in FIG. 3C, at 120 ml). The systolic phase termination coincides with ECG “T” wave's return to baseline coinciding with the smallest ventricular volume. The volume change from maximum to minimum designates the stroke volume (SV).

LV filling is the combination of multiple features such as LA contributions, valve (e.g., mitral valve (MV) and/or aortic valve function), and myocardium size, compliance and function. The following list is not intended to be all inclusive but only provides some appreciation for the complexity involved during the diastolic function and initial LV filling flow development. For example, LA considerations can include LA compliance, volume, contractile strength and preload. The MV's effects can include its ability to open and allow flow without tissue or structural resistance associated with calcification, fibrosis, or mobility issues affecting orifice availability, as well as possible leak or regurgitation. The LV myocardial effects can include rate of LV pressure recovery associated, myocardial relaxation coordination, ventricular compliance, and post systolic residual LV volume and pressure. Additional volume contributions occur because of aortic valve insufficiency and during the atrial systolic phase.

For Normal and Grade 1 diastolic dysfunction (ASE Guidelines), LV VCs correlate well with LVIT Doppler. For example, FIGS. 4A-4B are an illustrative graph (FIG. 4A) and a photo of (FIG. 4B) of a volume curve overlaid with left ventricular inflow tract (LVIT) Doppler aligned based on ECG. However, for more complicated or advanced diastolic dysfunction, LVIT Doppler classification is insufficient based at least on possible MV contributions. Accordingly, the present invention provides a method using VCs that is applicable across a range of Diastolic functional levels.

### Volume Curve Calculations and Measurements

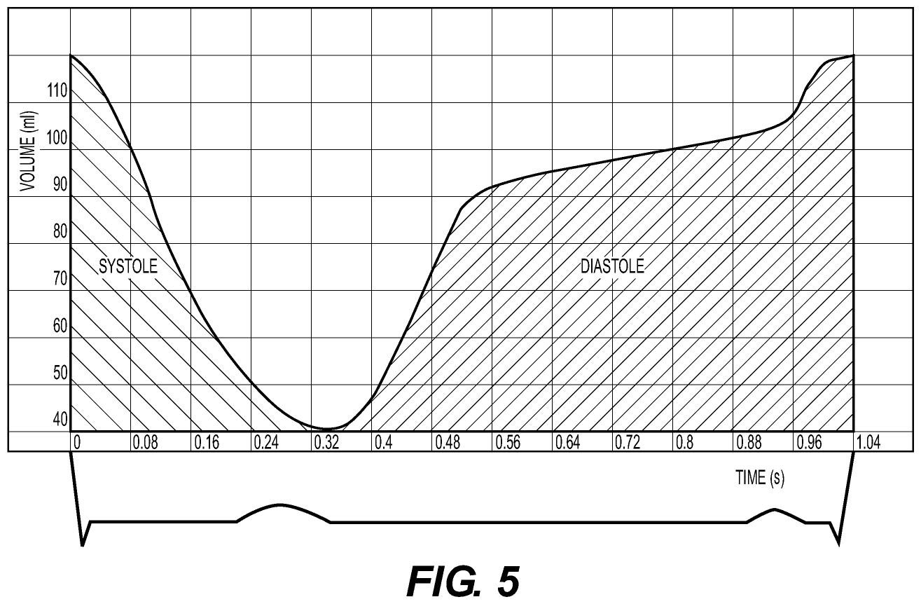

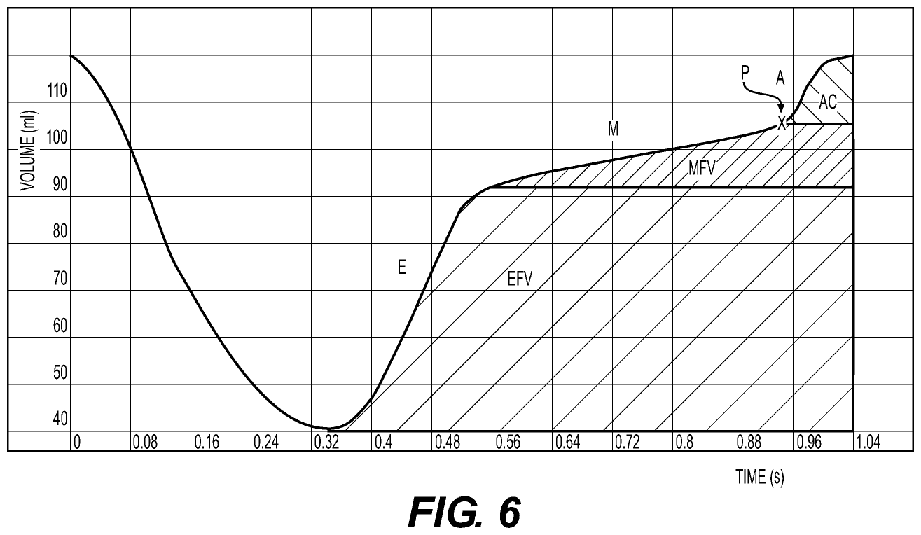

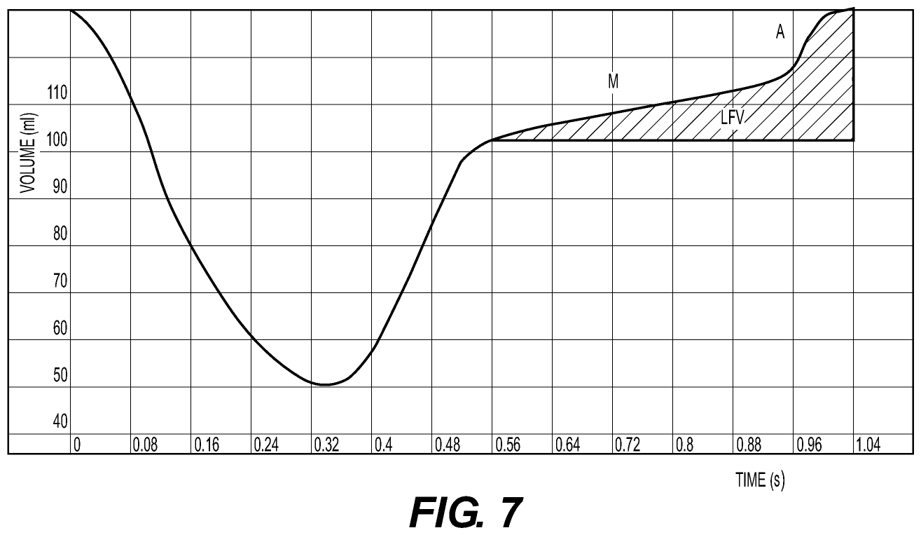

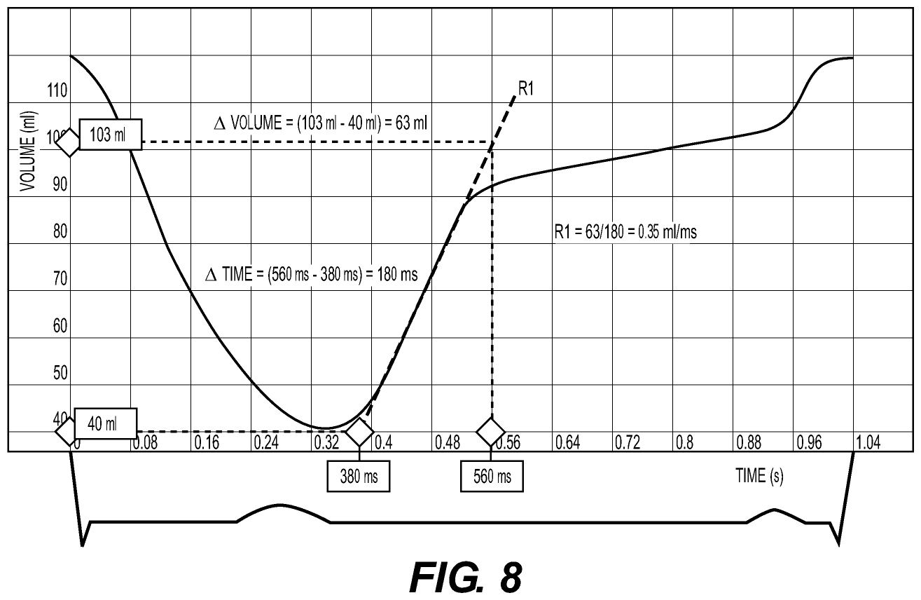

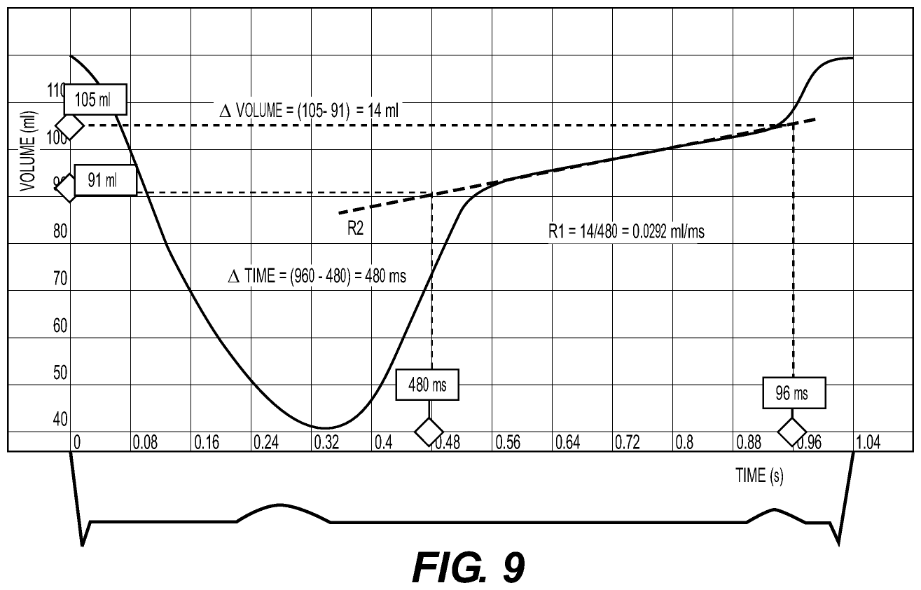

FIGS. 5-10 provide exemplary volume curves to illustrate components, intervals, and calculations useful according to the present application. In particular, FIG. 5 is an exemplary normal cardiac volume-time curve aligned with an exemplary ECG and showing systole and diastole. FIG. 6 is an exemplary cardiac volume-time curve with the early filing phase (E), the intermediate filling phase (M) and the atrial filling phase (A) of the diastolic phase labeled. FIG. 6 also includes shading to illustrate the early filling (EFV), mid-filling (MFV), and atrial contribution (AC) to filling during diastole. Point X in FIG. 6 indicates the ventricular volume (P) at the termination of the passive filling phase and the initiation of the atrial contraction phase. FIG. 7 is an exemplary cardiac volume-time curve with shading to illustrate the late filling (LFV), corresponding to the intermediate filling phase and the atrial filling phase, during diastole. FIG. 8 is an exemplary cardiac volume-time curve illustrating calculation of filling rate (R1) during an early filling phase. FIG. 9 is an exemplary cardiac volume-time curve illustrating calculation of filling rate (R2) during a mid-filling phase. FIG. 10 is an exemplary cardiac volume-time curve illustrating calculation of R1, R2, early filling volume (E), and filling volume (FV).

FIG. 5 first illustrates a normal volume curve aligned to an ECG (tracing under graph). As noted previously, the VC of FIG. 5 starts at the peak of the R-wave—at end-diastolic volume (EDV). Systole is represented by the initial downward slope of the curve until the end-systolic volume (ESV) is reached. Diastole follows and includes several phases, which are discussed with respect to FIG. 6.

FIG. 6 displays the major components comprising and encompassing all of diastole. As shown in FIG. 6, diastolic volume can be divided into the Early Filling Volume (E, EFV), occurring during initial ventricular filling corresponding to the Doppler LVIT E-wave (FIG. 4B), and Mid Filling Volume (M, MFV). The designation point of E termination and M origination is created when the flow rate changes during the low flow or mid filling phase transition. Further, as shown in FIG. 7, a Late Filling Volume or LFV can be identified by combining MFV with the atrial component (AC in FIG. 6). Volume and filling rate changes are indicative of intra and extra cardiac features including compliance, relaxation rate and volume interactions between the contributors.

FIGS. 8-10 illustrate calculation of filling rates during early and mid filling (R1 and R2 respectively). FIG. 8 is an exemplary cardiac volume-time curve illustrating calculation of filling rate (R1) during an early filling phase. FIG. 9 is an exemplary cardiac volume-time curve illustrating calculation of filling rate (R2) during a mid-filling phase. FIG. 10 is an exemplary cardiac volume-time curve illustrating calculation of R1, R2, early filling volume (E), and filling volume (FV).

The R1:R2 Ratio forms the initial relationship for LV VC evaluation. By comparing the passive filling phase relationship between the early (R1) and mid (R2) filling rates, the R1:R2 Ratio provides insight into the compliance relationships and pressure equalization rates during the passive filling phase between the LA and LV. R1 is equivalent to the Doppler derived mitral valve “E” wave flow incorporating the flow period between the “D” and “F” points including the peak “E” velocity and deceleration components and R2 is equivalent to the Doppler “diastasis” or low-flow period. R2: R3 intercept provides a specific end-volume for LV passive filling and the initiation of atrial contraction contribution to LV filling volume.

As illustrated in FIG. 8, R1 can be calculated as a slope of the volume curve, or dV/Dt during early filling. For simplicity, the R1 line has been extended to the bottom of the graph. The line/graph border intersection point provides a y-axis volume coordinate of 40 ml and an x-axis time coordinate of 380 ms. A second point is then chosen. In this example, the point provides a y-axis volume of 103 ml and an x-axis time of 560 ms. R1 is therefore calculated as the volume difference divided by the time difference as 63/180, or 0.35 ml/ms

Similarly, R2 can be calculated as a slope of the volume curve during the mid-filling phase (FIG. 9). In this example the A Volume is 14 ml and the A Time is 480 ms yielding a slope of 0.0292 ml/ms. R2=0.0292.

This example produces an R1:R2 Ratio of 11.99 (FIG. 10). In general, a higher R1:R2 Ratio is better, but one should further consider the atrial component, as discussed below.

### Volume Curve Classification

As noted previously, the use of VC allows evaluation of the many possible factors affecting diastolic filling. Accordingly, using the present methods and systems, numerous features of the VC can be considered in assessing diastolic function. This is because the VC offers much more than just passive filling rate evaluation. Calculations involving early filling volume (E), filling volume (FV), passive filling volume (P), and/or the atrial contribution (A) can be made and combined to provide diastolic performance differentiation and classification.

The Early Filling Volume (E) is the volume that occurs until mid filling starts or a point at which there is a transition in the curve from R1 to R2. It is determined at the intersection of VC and R2. The point of interception of R1 and R2 (E*) can provide an alternative to E when E is poorly defined. E* is at the intercept of slope lines R1 and R2 while E is at the first common point of the VC and R2 representing the initiation of mid LV filling. E and E* will often be very similar and in most cases can be used interchangeably without adversely affecting the method outcome. It is the volume transfer during the Doppler “E” wave duration (D-F). This value is independent of R1/R2 and a representation required when evaluating early diastolic performance. The BSA indexed Initial Filling Volume (Ei) more accurately represents E normalizing for body habitus while Ei % additionally normalizes for FV.

Filling Volume (FV) is the difference between end-systolic ventricular and end-diastolic volumes. Stroke Volume (SV) measurement will be similar to FV in the absence of abnormal volume contributions such as when significant aortic and/or mitral regurgitation are present depending on the method for calculating SV. The continuity based Doppler method (Qs) only measures the Forward cardiac output for SV. When significant aortic and/or mitral regurgitation are present, 3D volume renderings of the LV volume must be used. The FV includes contributions from both forward cardiac output, and retrograde, aortic and mitral insufficiencies. The Filling Volume indexed to body surface area (BSA) (FV/BSA (FVi) more accurately represents FV normalized for body habitus.

The R1:R2 Ratio can remain the same across various disease states independent of ventricular output and atrial contraction contribution (AC). The R1:R2 provides the core differentiator between various diastolic pathologies with R2 comparison being a missing component from previous published measures. R2 represents the normalization of LV passive filling providing an indicator of the compliance relationship between the LA and LV. According to the present methods, the observed normal R1:R2 range is about 4 to about 16 with the 111 patients studied.

As explained below, higher R1:R2 values without a significant AC indicate either poor ventricular compliance or a reduction in atrial contractility, while lower values indicate poor early filling often related to poor LA preload seen with hypovolemia or LV relaxation abnormalities.

As further explained, the present invention considers additional features, including at least one of the early filling volumes (E) and atrial contributions (A). As used herein (E/FV) or (Ei/FVi) or (Ej/FVj) is the ratio defining the balance of early and total filling contributions. BSA indexing or BMI indexing is not required but provides normalized values. Normal values for E/FV range from 0.50 to 0.70. Further, the BSA corrected Ei can be indexed to the FV resulting in Ei % ranging from 50 to 70%. Higher values differentiate poor later volume change associated with reduced LV compliance while lower values suggest poor ventricular relaxation dynamics or poor LA preload seen with hypovolemia. Similarly, the atrial contribution can be represented as A/FV, Ai/FVi (atrial contribution (A) and FV indexed to BSA corrected, or Ai % (Ai indexed to FV).

Volume curves can have similar appearances with significantly different ventricular outputs and clinical presentations. Differentiating between Normal Ventricular Output (NVO) and Low Ventricular Output (LVO) is Step 1 of the Decision Flowchart (FIG. 1B). Step 2 guides diagnosing diastolic performance using the relationships depicted in FIG. 2.

The first diastolic classification, as illustrated in FIG. 2, is a Normal diastolic function. Normal function corresponds to R1:R2 greater than about 4 and less than about 16, an E % of greater than about 50% and less than about 70%, and an A % of greater than about 25%. An example normal VC is shown in FIG. 5

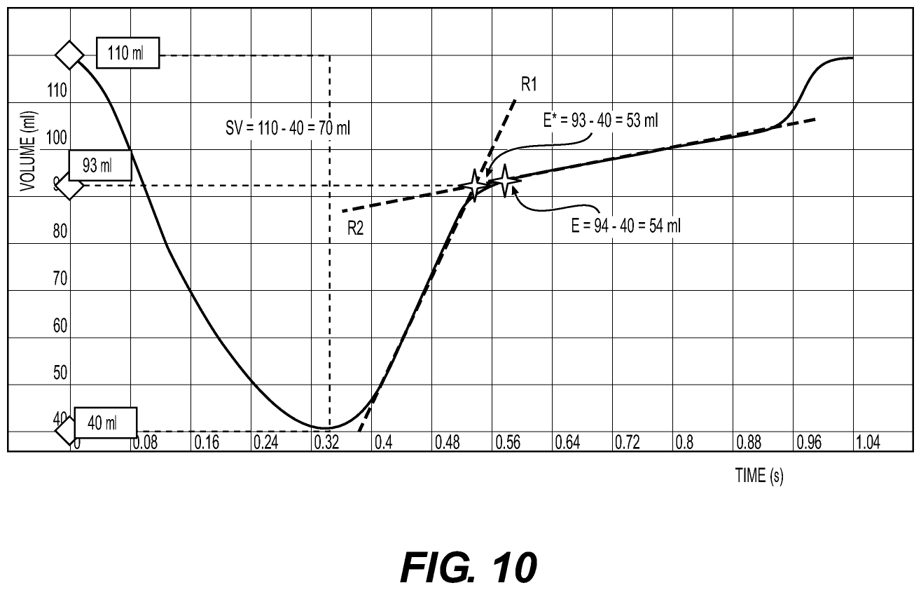

The second classification includes early enhanced filling. Early enhanced filling can be one of two types. Early enhanced can correspond to an R1:R2 ratio of greater than about 4 and less than about 16, an E % of greater than about 50% and less than about 70%, and an A % of less than about 25%, which is referred to as type 2 herein. Alternatively, an early enhanced classification can correspond to an R1:R2 ratio of less than about 16, and an E % of greater than about 70%, which is referred to as type 1 herein. An exemplary early enhanced VC is shown in FIGS. 11A and 11B.

As noted above, the early enhanced VC classification can occur with two types. Each type demonstrates smaller than normal volume enhancement during the late filling phase from which R2, R3, and P are measured and A is calculated, the atrial component contributing little to the FV. Most ventricular filling occurs early indicating rapid equalization of pressures and compliance between the LA and LV (FIGS. 11A and 11B). This group represents the extreme upper limit of normal requiring clinical correlation to differentiate early constrictive/restrictive pathology, poor LA contractility, uncompensated LV end-diastolic pressure (LVEDp) elevation or any condition reducing late volume transfer increasing the following cycle's LA preload.

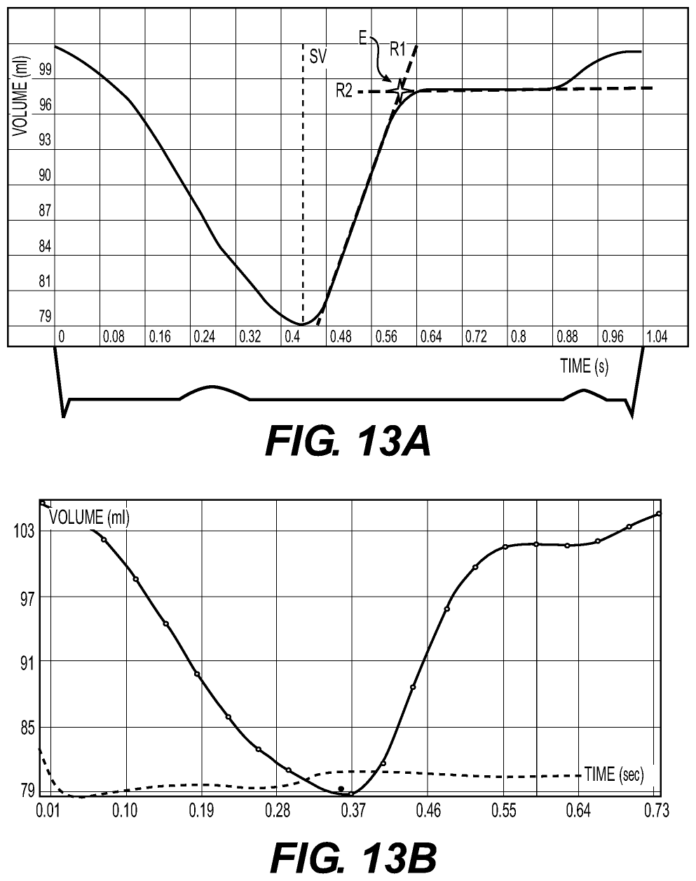

A third classification is a mid-enhanced classification. The mid-enhanced classification corresponds to an R1:R2 ratio of less than about 16, an E % of less than about 50%, and an A % of less than about 30%. An exemplary mid-enhanced filling VC is found in FIGS. 14A and 14B.

The mid-filling enhanced VC classification refers to the filling volume following early filling. This class represented the largest single class of study subjects. The majority presented with echo/Doppler measures supporting hypovolemia with IVC resting collapse, prolonged IRT, lower than expected E/e′, D-E slopes less than 800 cm/s, E/A ratios <0.8, E-wave <50 cm/s and pulmonary venous waveforms with prominent “D” and absent or blunted “S” waves. Most with dilated LA and pulmonary veins without evidence of significant MV disease during a resting echocardiogram. The flattened curve (FIG. 14) represents a decrease in cardiac reserve. As heart rate increases with activity, the diastolic filling period shortens, thereby decreasing LV filling. Patients with this classification commonly complain of lack of energy, dyspnea with minimal activity, and constant need to rest.

A fourth classification includes atrial enhanced filling. Atrial enhanced filling can be one of two types. One type of atrial enhanced filling classification, referred to as type 1 herein, corresponds to an R1:R2 ratio of less than about 16 and greater than about 4, an E % of less than about 50% and an A % of greater than about 30%. A second type of atrial enhanced filling classification corresponds to an R1/R1 of greater than about 16, an A % of greater than about 30%, and an MVOi of less than about 2200 ml.

The atrial enhanced classification is a late enhancement form resulting from the atrial contraction presenting with an R1:R2<16.00 primarily because of near diastasis producing a low R2 value. This demonstrates less than optimal filling during the passive filling that is compensated for by an aggressive atrial contraction to “top-off” the LV. This can be seen in hypovolemia. Normal balance between Passive and Active Filling is 60/40. The low R2 slope alone cannot be used to differentiate classes. This feature may be present in aerobically conditioned athletes and young adults representing excellent compliance features but is also a component with restrictive/constrictive disease.

A fifth classification includes an aerobic enhanced classification. The aerobic enhanced classification corresponds to an R1:R2 ratio of greater than about 16, an Ai % of greater than about 30%, and an MVOi of greater than about 2200 ml.

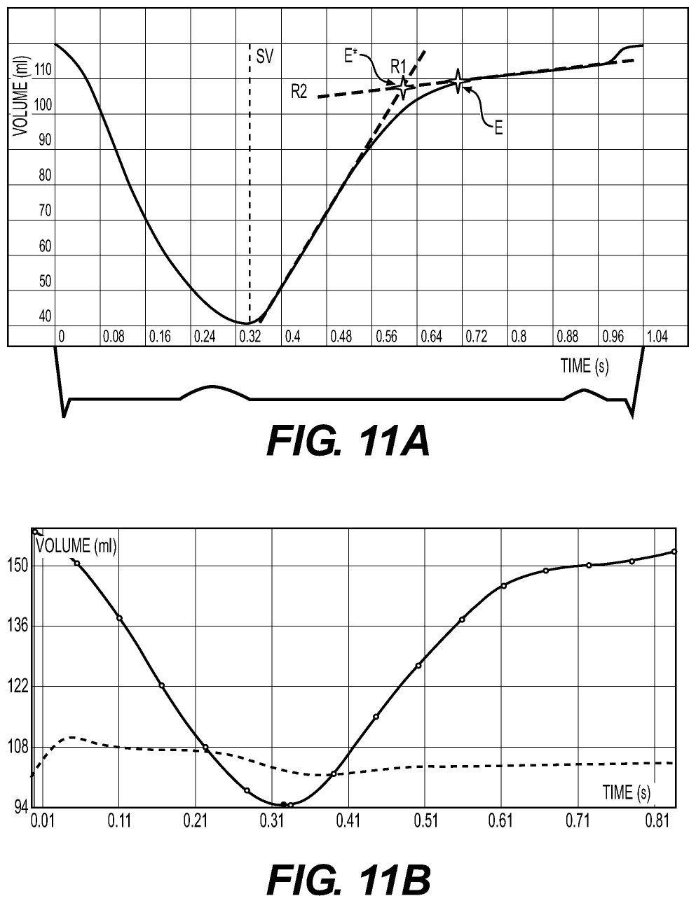

A sixth classification includes a delayed filling classification. Delayed filling corresponds to an R1:R2 ratio of less than about 4, and an E % of greater than about 50%. An example delayed filling volume curve is found in FIGS. 12A and 12B.

The delayed filling VC classification has an R1:R2 Ratio less than normal but maintains a normal BSA corrected late volume. Representing the second highest populated study class the majority presented with greater than 100 ms IRT measurements suggesting poor ventricular relaxation dynamics. (FIG. 12). The most obvious differences are the decreased R1 causing the abnormally low R1:R2 with a normal or near normal E %. At times, the R1 and R2 slopes can appear as nearly a single line. Normally the LV pressure drop occurs within 80 ms, but when delayed because of dyssynchrony of ventricular relaxation, the E will be delayed affecting cardiac reserve. The further to the right on the curve the E occurs, the shorter the time between the atrial contraction. Once these two components fuse then any increase in heart rate will reduce FV, and subsequently SV, leading to a state of exacerbation. The enhanced volume contribution during R2 can also be a result of continued LA filling from the pulmonary veins during diastole with the LA compliance being less than the LV. Rather than the LA being able to simply absorb the volume as seen when the R2 slope approaches 0, it is directly passed through to the LV.

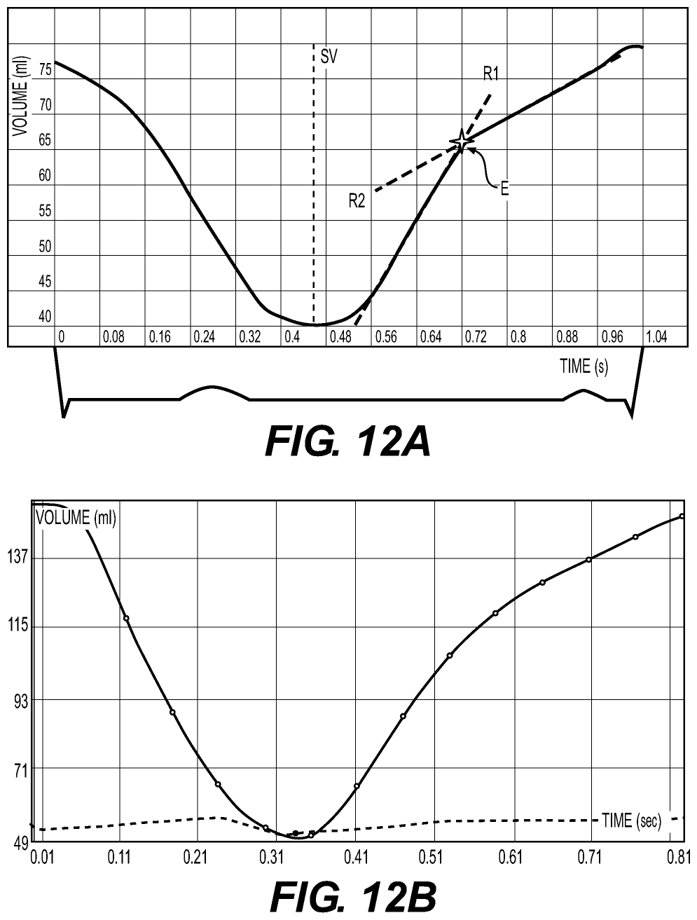

A seventh classification includes a restrictive/constrictive classification. The restrictive/constrictive classification corresponds to an R1:R2 ratio of greater than about 16, and an A % of less than about 30%. An exemplary restrictive/constrictive VC is found in FIGS. 13A and 13B.

The restrictive/constrictive VC classification demonstrates rapid early filling with very little additional filling volume later. This differentiates them from the rapid early filling observed within normal patients.

Finally, an eighth classification includes a hypovolemic classification that corresponds to an R1/R2 of less than about 4, an E % less than about 50%, and an A % greater than about 30%.

As VC analysis expands into young adult and athletic populations, differentiation between mid, AC, and mixed enhancement may be required.

The flowchart/decision tree of FIG. 1B graphically represents a method of classifying diastolic function in accordance with some embodiments. For example, if the cardiac data corresponds to R1/R2 less than or equal to 16.00 at decision point 30, E % not greater than or equal to 70% (i.e., E % less than 70%) at decision point 32, E % not greater than or equal to 50% (i.e., E % less than 50%) at decision point 34, and A % not greater or equal to 30% (i.e., A % less than 30%) at decision point 36, the classification would be mid-enhanced filling. In another path along same branch up until decision point 36, if A % is greater or equal to 30% at decision point 36, and R1/R2 not greater than or equal to 4 (i.e., R1/R2 less than 4) at decision point 38 the classification would be hypovolemic. In another path along same branch up until decision point 38, if R1/R2 is greater than or equal to 4 at decision point 38 the classification would be atrial enhanced filling (type 1). This is one of two different paths in the flow chart that lead to atrial enhanced filling with different paths corresponding to different types.

Backing up to decision point 34, for cardiac data corresponding to E % greater than or equal to 50% at decision point 34 and R1/R2 greater than 4.00 at decision point 40, the classification would be delayed filling. For another path along the same branch up until decision point 40, for cardiac data corresponding to R1/R2 not greater than 4.00 (i.e., R1/R2 less than or equal to 4.00) at decision point 40, and A % greater than or equal to 25%, the classification would be normal. For another path along the same branch up until decision point 42, for cardiac data corresponding to A % not greater than or equal to 25% (i.e., A % less than 25%) at decision point 42, the classification would be early enhanced (type 2). Notably, this is one of two paths in the flowchart that lead to the early enhanced classification with the different paths corresponding to different types. Backing up to decision point 32, if the cardiac data corresponds to E % greater than or equal to 70% at decision point 32, the classification would be early enhanced (type 1).

Backing up to decision point 30, for cardiac data corresponding to R1/R2 not less than or equal to 16.00 (i.e., R1/R2 greater than 16.00) at decision point 30, and A % not greater than or equal to 30% (i.e., A % less than 30%) at decision point 44, the classification would be restrictive/constrictive cardiomyopathy. Along same branch up until decision point 44, for cardiac data corresponding to A % greater than or equal to 30% at decision point 44, and minute ventricular output BSA indexed (MVOi) greater than or equal to 2200 ml at decision point 46, the classification would be aerobic enhanced. Along same branch up until decision point 46, for cardiac data corresponding to minute ventricular output BSA indexed (MVOi) not greater than or equal to 2200 ml (e.g., MVOi less than 220 ml) at decision point 46, the classification would be atrial enhanced filling (type 2), which is the second path to atrial enhanced filling.

In the example, the following equation was used to properly distribute the study population of 111 subjects into each of the eight Volume Curve classes based on the descriptions previously provided.

Expressions for classes based on E and A normalized to FV (E % and A %) classes based on E and A normalized to FV and indexed to BSA (Ei % and Ai %) appear below:

```
  VC Class: IIf([R1/R2]<=16, IIf([E%]>=0.7,“Early Enhanced”, (IIf([E%]>=0.5,
(IIf([R1/R2]<4,“Delayed Filling”, IIf([A%]>=0.25,“Normal”,“Early Enhanced”))),
IIf([A%]>=0.3, IIf([R1/R2]>=4,“Atrial Contraction Enhanced”,“Mid Enhanced”),“Mid
Enhanced”)))), IIf([A%]<0.3,“Restrictive/Constrictive Disease”,
IIf([SVOi]>=2200,“Aerobic Enhanced”,“Atrial Contraction Enhanced”)))
  VC Class: IIf([R1/R2]<=16,IIf([Ei%]>=0.7,“Early
Enhanced”,(IIf([Ei%]>=0.5,(IIf([R1/R2]<4,“Delayed
Filling”,IIf([Ai%]>=0.25,“Normal”,“Early
Enhanced”))),IIf([Ai%]>=0.3,IIf([R1/R2]>=4,“Atrial Contraction
Enhanced”,“Hypovolemic”),“Mid Enhanced”)))),IIf([Ai%]<0.3,“Restrictive/Constrictive
Disease”,IIf([SVOi]>=2200,“Hyper Atrial Contraction”,“Atrial Contraction
Enhanced”)))
```

In the expressions above, “IIf” is a function, also referred to as “Immediate If”, having the form “IIf(expr, truepart, falsepart)”, where expres is the expression to be evaluated, truepart is the value or expression returned if expr is True, and falsepart is the value or expression returned if expr is False.

One of ordinary skill in the art, in view of the present disclosure, will appreciate that embodiments for cardiac function evaluation may employ a flowchart having a different structure that than of FIG. 1B, may employ a different classification equation than that presented above, and/or may employ different cutoffs for various criteria than those employed in the flow chart of FIG. 1B and the Table of FIG. 2 and still fall within the scope of the present invention. For example, in some embodiments the cutoffs for E % and A % could vary by +/−10%, and the R1/R2 upper cutoff could range into the low 20s (e.g., 23).

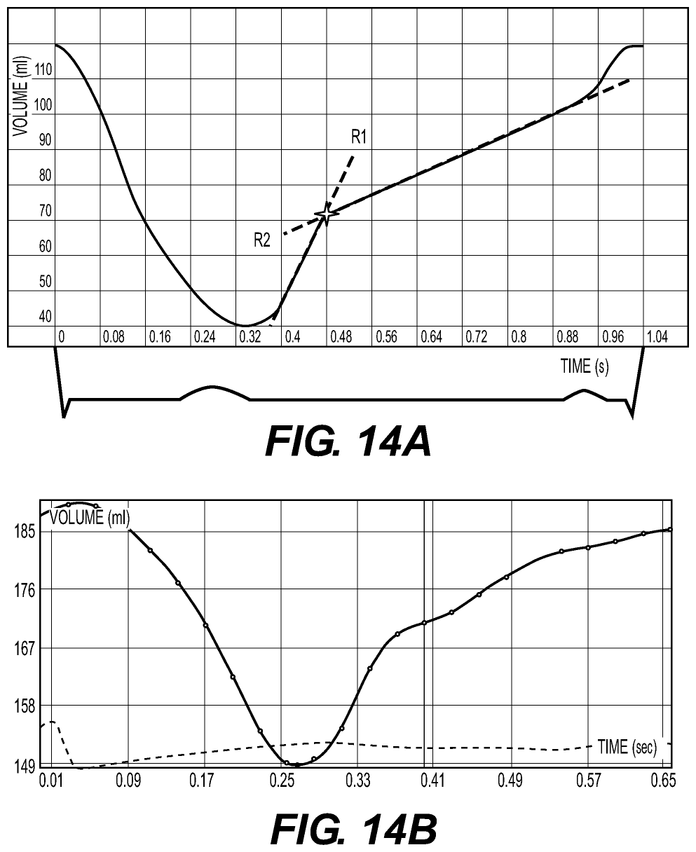

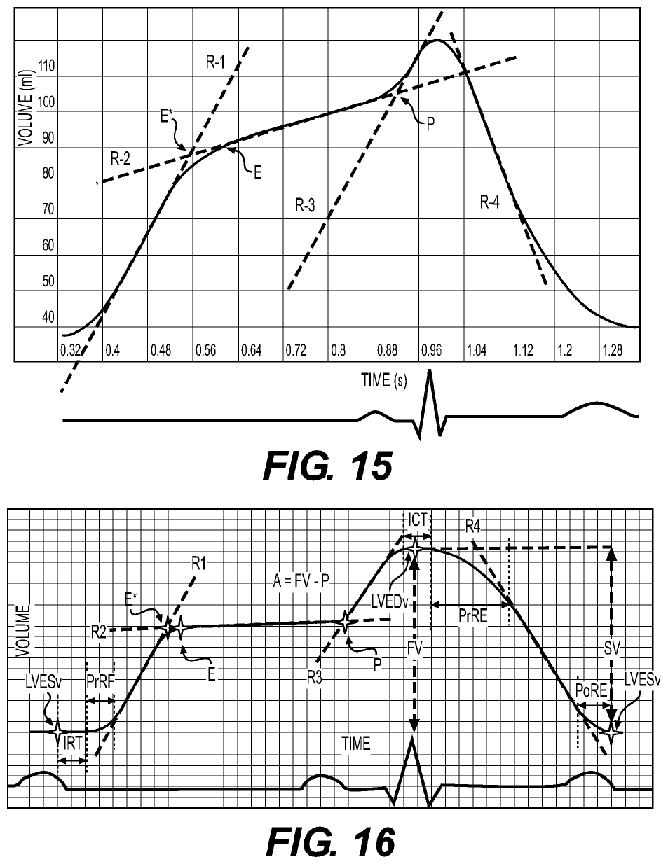

FIGS. 15 and 16 illustrate additional features and calculations based on VCs that may be used in the present methods. FIG. 15 is a graph illustrating various measurements and calculations relating to an exemplary volume curve. FIG. 16 is a graph illustrating various measurements and calculations relating to a complete diastolic-systolic volume curve capture. In particular, FIGS. 15 and 16 illustrate various previously described values (e.g., R1, R2, and E) and also illustrate exemplary methods for calculation of the atrial contribution (A). As shown, an atrial filling rate (R3) can be calculated as the slope of the volume curve during the atrial contraction phase. The initiation of the atrial contraction can be identified as point P. P can be determined as either the intersection of the intermediate slope line R2 with a slope line of the most rapid filling during the atrial contraction, R3, or as the point of VC separation from slope line R2 (P*), or as the point of initial intersection of the VC with R3 (P3). See FIG. 15. The atrial contribution (A) can be determined by subtracting the P measurement from the FV with P representing the total passive volume of the FV. As such, A=FV-P (or FV=P* or FV-P**). Either technique will produce similar results by both A volume calculation and VC classification with the preferred method using A=FV-P because of the lower variability of P.

FIG. 17 is a diagram illustrating an embodiment of a system 100 for performing cardiac function assessment. The system 100 can be embodied as a central processing unit 110 (processor) in communication with a database 120. The processor 110 can include, but is not limited to, a processing circuitry, a computer system, a server, a personal computer, a cloud computing device, a smart phone, or any other suitable device or circuitry programmed to carry out the processes disclosed herein. Still further, the system 100 can be embodied as a customized hardware component such as a field-programmable gate array (“FPGA”), an application-specific integrated circuit (“ASIC”), embedded system, or other customized hardware components without departing from the spirit or scope of the present disclosure. In some embodiments, the system 100 or the processor 110 is part of the imaging modalities that capture one or more images during one or more complete cardiac cycles. In some embodiments, the system 100 or the processor 110 can communicate with the imaging modalities to obtain the images captured by the imaging modalities. It should be understood that FIG. 17 is only one potential configuration, and the system 100 of the present disclosure can be implemented using a number of different configurations.

The database 120 could include images and/or image datasets comprising images obtained from different imaging modalities including, but not limited to echocardiography, computed tomography (CT), and magnetic resonance imaging (MRI). The database 120 could include volume datasets, images or models reconstructed by the imaging modalities. The database 120 could store one or more three-dimensional representations (e.g., three-dimensional images, three-dimensional reconstructed models) of an imaged heart, and the system 100 could operate with such three-dimensional representations. The processor 110 executes system code 130 which assesses cardiac function using the images from the database 120 and/or from different imaging modalities described therein. The database 120 can further include one or more outputs from various components of the system 100 (e.g., outputs from a phase identification engine 132, a ventricular filling rate determination engine 134, a ventricular filling volume determination engine 136, a diastolic function classification engine 138, a systolic function determination engine, and/or other components of the system 100).

The system 100 includes system code 130 (non-transitory, computer-readable instructions) stored on a computer-readable medium and executable by the hardware processor 110 or one or more computer systems or one or more imaging modalities. The system code 130 can include various custom-written software modules that carry out the steps/processes discussed herein, and can include, but is not limited to, the phase identification engine 132, the ventricular filling rate determination engine 134, the ventricular filling volume determination engine 136, the diastolic function classification engine 138, systolic function determination engine, and/or other components of the system 100. The system code 130 can be programmed using any suitable programming languages including, but not limited to, C, C++, C#, Java, Python, or any other suitable language. Additionally, the system code 130 can be distributed across multiple computer systems in communication with each other over a communications network, and/or stored and executed on a cloud computing platform and remotely accessed by a computer system in communication with the cloud platform. The system code 130 can communicate with the database 120, which can be stored on the same computer system as the system code 130, or on one or more other computer systems in communication with the system code 130.

It is noted that the phase identification engine 132 can identify a diastolic phase of the heart using an images captured during one or more complete cardiac cycles. The phase identification engine 132 can further identify in the diastolic phase an early filling phase, an intermediate filling phase, and an atrial contraction phase. The phase identification engine can identify a systolic phase during one or more complete cardiac cycles dependent on capture trigger technique using either the ECG R-wave or T-wave. R-wave triggering will require a minimum 2 beat capture to provide sufficient data points to extract a single complete cycle. A T-wave to T-wave capture can capture the related diastolic and systolic phases in a single cycle. The ventricular filling or ejection rate determination engine 134 can determine a left ventricular filling rate (R1) during the early filling phase, a left ventricular filling rate (R2) during the intermediate filling phase, a left ventricular filling rate (R3) during the atrial contraction phase and a rapid ejection rate (R4) phase during systole. The ventricular volume change determination engine 136 can determine a first left ventricular filling volume (E) during the early phase, a second left ventricular filling volume (P) prior to the atrial contraction phase, a total filling volume from which the A volume can be determined. The diastolic function classification engine 138 can classify a diastolic function classification based on R1, R2, E and A.

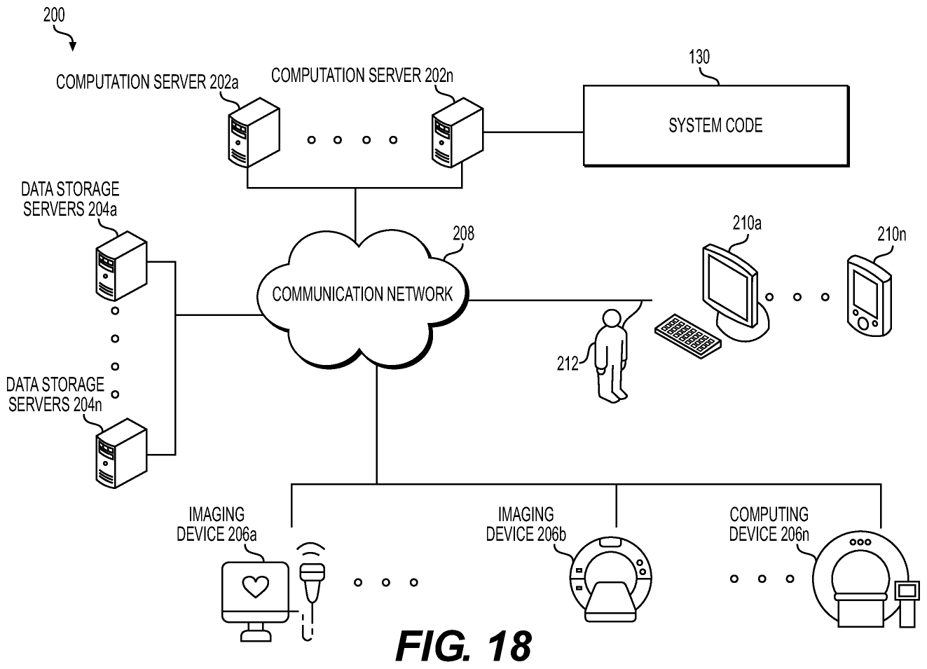

FIG. 18 is a diagram illustrating hardware and software components capable of being utilized to implement a system 200 of the present disclosure. The system 200 can include a plurality of computation servers 202a-202n having at least one processor (e.g., one or more graphics processing units (GPUs), microprocessors, central processing units (CPUs), tensor processing units (TPUs), application-specific integrated circuits (ASICs), etc.) and memory for executing the computer instructions and methods described above (which can be embodied as system code 130). The system 200 can also include a plurality of data storage servers 204a-204n for storing images and/or the system code 130. Computing devices 210a-210n can include, but it not limited to, a computer, a laptop, a smart telephone, and a tablet to access and/or execute the system code 130 and communicate with imaging devices 206a-206n. The imaging devices 206a-206n can provide images of heart with physical and/or functional structures. The imaging devices 206a-206n can include, but are not limited to, imaging device/systems for echocardiography, computed tomography (CT), and magnetic resonance imaging (MRI) or other imaging modalities. The computation servers 202a-202n, the data storage servers 204a-204n, the imaging devices 206a-206n, and the computing devices 210a-210n can communicate over a communication network 208. Of course, the system 200 need not be implemented on multiple devices, and indeed, the system 200 can be implemented on a single device (e.g., a personal computer, server, mobile computer, smart phone, or an imaging device/system etc.) without departing from the spirit or scope of the present disclosure.

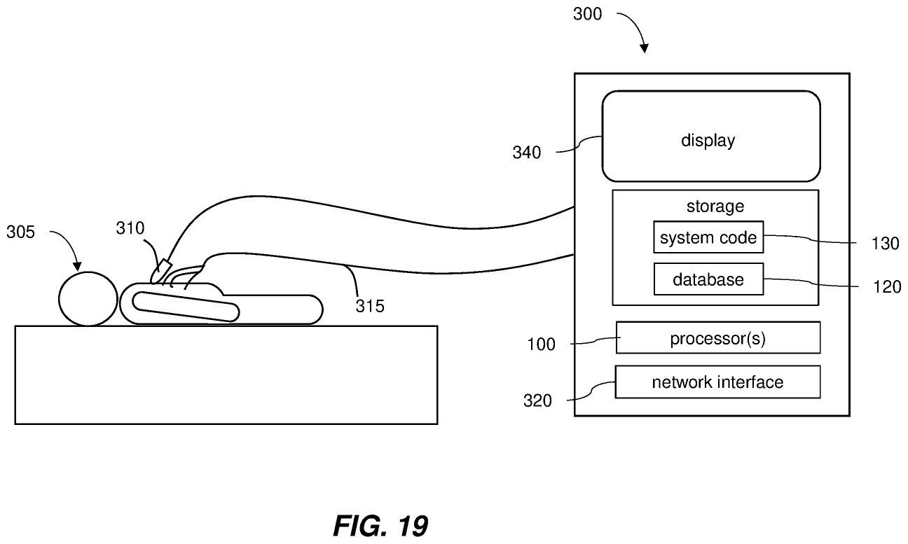

For example, FIG. 19 schematically depicts an integrated system 300 that both images a heart of a patient/subject 305 and performs diastolic function classification in accordance with some embodiments. System 300 includes imaging instrumentation that includes an ultrasound probe 310. In some embodiments, the system also includes electrocardiogram (ECG) imaging instrumentation 315 to aid in timing of image acquisition. In other embodiments, imaging instrumentation may employ other imaging techniques. System 300 also include at least one processor 100 for implementing system code 130. System 300 may store all system code 130 (e.g., in storage 320) or may obtain part or all of the system code from another system via a connection to a communication network using a network interface 320 of the system. System may also include one or more databases 120 to store cardiac imaging data. In some embodiments, the cardiac imaging data may also or alternatively be stored outside of the system 300 and accessed via a communication network. In some embodiments, the system includes a display 340 for displaying images and information to a user.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to the respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

By being able to quantify diastolic dysfunction by various parameters or weighted features, a more precise measure of a specific disease state can be measured. The diastolic filling period (DFP) is defined as a difference between the cycle duration and systolic duration. Using time or heart rate, a relationship including all components of cardiac function is developed. The percent of initial filling volume helps to identify individuals with poor active atrial contributions such as with atrial fibrillation. The stroke volume (SV) feature will correct for changes in volume loading. There may also be iterations that involve the use of multiple factors in various combinations to describe the function.

The present system and method are useful in the diagnosis, stratification of diastolic function and treatment strategies for diastolic heart failure across multiple imaging modalities including CMR, CCT, CNM, and echo or any volume rendering method, providing a global measure of diastolic performance.

One value of this measure is in the quantification of complex relationships of ventricular filling. Systems and methods according to the teachings of the present invention are useful as exemplified such will be beneficial in clinical assessment of heart failure patients for differentiation of causality and direction of care qualifying and quantifying diastolic performance. Examples include physiologic testing especially endurance training and cardiac recovery; women's medicine to better understand hormone protection and HF; titration of medical therapy for the treatment of hypertension; scar load post myocardial infarct effect on ventricular compliance and quantifying the cardiac benefit of rehab; interventional planning for therapies and corrective procedures involving the heart, the effect of hypertension on ventricular compliance; as well as other restrictive and constrictive cardiomyopathies; and optimizing device therapies such as biventricular pacemakers or carotid body stimulators. Further, benefits will be seen in pharmaceutical management for diuretics and beta blockers as well as any diastolic focused medical therapies. Any study or study of cardiac function will benefit from the ability to specifically describe the diastolic relationship in a numeric form.

### Readmission Rate Prediction and Identification of Patients at Elevated Risk of Readmission

For many medical conditions, including heart failure, it would be useful to be able to predict the likelihood for hospital readmission within a relatively short time period, such as 30 or 90 days. Such predictions may help guide care, including for example, more intensive outpatient monitoring, changes to treatment such as medication, or continuing or initiating a hospital stay until the likelihood for readmission has decreased. As such, the present disclosure provides methods for predicting readmission in patients with cardiac conditions, particularly heart failure. The methods and devices or systems employing such methods can utilize volume curve data, as provided above combined with other parameters, as explained further.

A study (INDEX) was performed at Advent Health System, Florida Hospital, Orlando Florida. The study included one hundred subjects from a Heart Failure (HF) database combined with eleven age-appropriate controls whose data was reviewed retrospectively for admissions with 30-days and 90-days. The cohort characteristics appear below in

TABLE 1
Table 1 - Cohort Characteristics

| | Whole | Admitted within 30 days | | | Admitted within 90 days | | |
|---|---|---|---|---|---|---|---|
| | Cohort | Yes | No | P- | Yes | No | P- |
| | (n = 111) | (n = 10) | (n = 101) | value | (n = 18) | (n = 93) | value |
| Sex Male (%) | 57(51.4) | 4(40.0) | 53(52.5) | 0.45 | 8(44.4) | 49(52.7) | 0.52 |
| | | | Mean ± SD | | | | |
| Age (years) | 67.4 ± 11.1 | 68.5 ± 8.5 | 67.3 ± 11.4 | 0.81 | 69.7 ± 8.9 | 66.9 ± 11.5 | 0.42 |
| BMI (kg/m2) | 29.4 ± 17.9 | 29.7 ± 7.6 | 29.4 ± 18.6 | 0.43 | 29.1 ± 6.3 | 29.5 ± 19.4 | 0.31 |
| BSA | 1.9 ± 0.24 | 1.9 ± 0.3 | 1.9 ± 0.2 | 0.69 | 1.9 ± 0.3 | 1.9 ± 0.2 | 0.60 |
| R1/R2 | 4.2 ± 4.4 | 2.4 ± 0.9 | 4.4 ± 4.6 | 0.052 | 3.5 ± 4.6 | 4.3 ± 4.4 | 0.052 |
| ASE (%) | | | | 0.82 | | | 0.37 |
| Normal | 32(28.8) | 2(20.0) | 30(29.7) | | 2(11.1) | 30(32.3) | |
| Grade 1 | 32(28.8) | 4(40.0) | 28(27.7) | | 7(38.9) | 25(26.9) | |
| Grade 2 | 24(21.6) | 2(20.0) | 22(21.8) | | 4(22.2) | 20(21.5) | |
| Grade 3 | 14(12.6) | 2(20.0) | 12(11.9) | | 3(16.7) | 11(11.8) | |
| Indeterminate | 9(8.1) | 0(0.0) | 9(8.9) | | 2(11.1) | 7(7.5) | |
| VC (%) | | | | 0.25 | | | 0.015 |
| Atrial Contraction | 3(2.7) | 0(0.0) | 3(3.0) | | 0(0.0) | 3(3.2) | |
| Enhanced | | | | | | | |
| Normal | 5(4.5) | 0(0.0) | 5(5.0) | | 0(0.0) | 5(5.4) | |
| Early Enhanced | 29(26.1) | 0(0.0) | 29(28.7) | | 0(0.0) | 29(31.2) | |
| Delayed Filling | 32(28.8) | 5(50.0) | 27(26.7) | | 8(44.4) | 24(25.8) | |
| Mid Filling | 40(36.0) | 5(50.0) | 35(34.7) | | 9(50.0) | 31(33.3) | |
| Enhanced | | | | | | | |
| Restrictive/ | 2(1.8) | 0(0.0) | 2(2.0) | | 1(5.6) | 1(1.1) | |
| Constrictive | | | | | | | |
| Disease | | | | | | | |
| ASE Group (%) | | | | 0.88 | | | 0.47 |
| Lower Grade | 64(57.7) | 6(60.0) | 58(57.4) | | 9(50.0) | 55(59.1) | |
| Higher Grade | 47(42.3) | 4(40.0) | 43(42.6) | | 9(50.0) | 38(40.9) | |
| VC Group (%) | | | | 0.029 | | | <0.001 |
| Normal | 37(33.3) | 0(0.0) | 37(36.6) | | 0(0.0) | 37(39.8) | |
| Abnormal | 74(66.7) | 10(100.0) | 64(63.4) | | 18(100.0) | 56(60.2) | |

ASE heart failure classification populated at least two subjects into each of four available gradings, specifically, Normal, Grade 1, Grade 2, and Grade 3, with subjects having the Intermediate Grade being excluded. Diastolic performance was considered a “Lower Grade” (n=64) of abnormality with “Normal’ or “Grade 1” and “Higher Grade” (n=47) of abnormality with “Grade 2” or “Grade 3”. VC classification was used to distribute the HF subjects with hospital admissions within thirty days following an echo procedure equally divided between 8 groupings: Restrictive Constrictive, Aerobic Enhanced, Atrial Contraction Enhanced, Mid Enhanced, Delayed Filling, Early Enhanced, Hypovolemic, and Normal. Volume curve (VC) classification using only VC features including filling rates and volumes of early and mid-passive filling, and atrial contraction components provided clear differentiation of 3 “Normal” (n=37) and 4 “Abnormal” (n=74) combinations, where the “Normal” grouping was found to include Atrial Enhanced, Normal, and Early Enhanced, and the “Abnormal” groups was found to include Mid Enhanced, Delayed Filling, Restrictive/Constrictive, and Hypovolemic. Extending the follow-up to ninety days yielded eighteen admissions with all but one contained within the same VC classifications and with ASE distribution similarly distributed across all five grades with the exception of Grade 1 accounting for seven subjects. The Indeterminate grade, accounting for one subject in 90 day and nine INDEX subjects total, was excluded because it does not differentiate between normal and abnormal ventricular ejection fraction, which is a requirement for Normal grade.

Cox-regression analysis, Kaplan-Meier curves and log rank test were used to compare the groups of normal and abnormal based on 3D echo generated volume-curve (VC) classification and the groups based on lower-grade and higher-grade in Echo/Doppler (ASE) classification. The results for the Cox regression analysis appear below in Table 2.

TABLE 2
Univariate Cox Regression Analyses - time to admission

| | Censored at 30 days | | Censored at 90 days | |
|---|---|---|---|---|
| | Hazard Ratio | P_{—} | Hazard Ratio | P_{—} |
| Variable | (95% CI) | value | (95% CI) | value |
| Sex Female | 1.60 [0.45-5.67] | 0.47 | 1.36 [0.54-3.45] | 0.52 |
| Age | 1.01 [0.95-1.07] | 0.75 | 1.02 [0.98-1.07] | 0.35 |
| BMI (kg/m2) | 1.01 [0.95-1.00] | 0.95 | 1.00 [0.97-1.03] | 0.95 |
| BSA | 1.94 [0.14-27.05] | 0.62 | 0.70 [0.1-5.0] | 0.72 |
| R1/R2 | 0.619 [0.36-1.07] | 0.08 | 0.93 [0.77-1.12] | 0.46 |
| ASE Group | 0.91 [0.26-3.23] | 0.89 | 1.37 [0.54-3.45] | 0.51 |
| Higher Grade | | | | |
| VC Group | | 0.021 | | 0.001 |
| Abnormal | | | | |

Note:
VC Group: log rank test of Kaplan-Meier

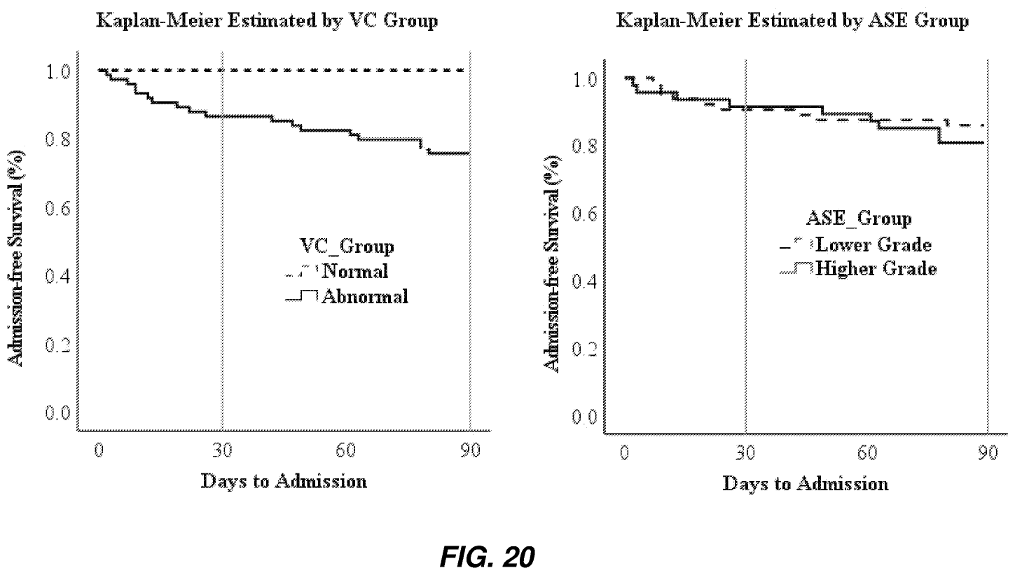

The results show the ASE groups demonstrated no significant differences with P-value=0.89 (Hazard Ratio (HR)=0.91, 95% Cl: 0.26-3.23) and P-value=0.51 (HR=1.37, 95% Cl: 0.54-3.45) for at risk of admission within 30-days and 90-days, respectively. In contrast, the VC groups showed significant differences with p-value=0.021 and p-value <0.001 for at risk of admission within 30 days and 90 days, respectively. With no admissions occurring within the Normal group, neither HR or Cls could be calculated. This is illustrated in the Kaplan-Meier curves for in FIG. 20, which show little difference between the Lower Grade and Higher Grade groups based on ASE grade, but show significant differences between the Normal and Abnormal groups based on VC classification.

Using data from the INDEX study in which each patient was evaluated using echocardiography, different methods were employed for assessing likelihood of hospital readmission. It is contemplated that the methods can be used to predict readmission or for outpatients, and/or the need for an initial admission due to potentially deteriorating conditions.

In some embodiments, performing a diastolic function classification of a patient's heart and determining whether that classification falls in the normal group (e.g., normal, atrial enhanced, and early enhanced) or the abnormal group (e.g., mid enhanced, delayed filling, restrictive/constrictive, and hypovolemic) enables a determination of whether the patient has a normal likelihood of readmission associated with the normal group or a higher or elevated likelihood of readmission associated with the abnormal group (see FIG. 20). In some embodiments, this enables efficient identification of heart failure patients at elevated risk of readmission. Identifying patients at elevated risk for hospital admission enables focusing of resources on the higher risk patients. In some embodiments,

In addition to the use of VC grouping, various expressions were employed and evaluated to determine the best method for assessing readmission probability. Expressions that incorporated VC data indexed to BSA or to BMI demonstrated good results. Specifically, a Moro index based on BSA (MI BSA), and a Moro index based on BMI (MI BMI), which both employ a R1/R2 Rank were best for assessing readmission probability. Expressions for a Moro index based on BSA (MI BSA), and a Moro index based on BMI (MI BMI) are below.

### MI BSA:

[R1/R2 Rank]*(IIf([Ai]>[Ei], ([Ei]/[Ai]), [Ai/Ei]))*[Ei/Pi]*([Ai]/([Ai]+[Mj]))*10, where “i” indicates value divided by the BSA

R1/R2 Rank_{indexed} to BSA:

$$IIf ( [ A i \% ] > = 0.2 , [ R 1 / R 2 ] , IIf ( [ R 1 / R 2 ] <= 16 , [ R 1 / R 2 ] , IIf ( [ R 1 / R 2 ] < 20 , ( 32 - [ R 1 / R 2 ] ) , 1 ) ) )$$

### MI BMI:

[R1/R2 Rank]*(IIf([Aj*5]>[Ej*3],([Ej]/[Aj),[Aj/Eij])*[Ej/Pj]*([Aj]/([Aj]+[Mj]))*10, where “j” indicates value divided by the BMI

R1/R2 Rank_{indexed to BMI}:

$$IIf ( [ A i \% ] > = 0.2 , [ R 1 / R 2 ] , IIf ( [ R 1 / R 2 ] <= 16 , [ R 1 / R 2 ] , IIf ( [ R 1 / R 2 ] < 20 , ( 32 - [ R 1 / R 2 ] ) , 1 ) ) )$$

In the expressions above, “IIf” is a function, also referred to as “Immediate If”, having the form “IIf (expr, truepart, falsepart)”, where expres is the expression to be evaluated, truepart is the value or expression returned if expr is True, and falsepart is the value or expression returned if expr is False.

The Moro index is also referred to as VC_{MI} herein. Analysis employing these expressions is referred to as VC_{MI} evaluation or Moro index (MI) evaluation herein. VC_{MI} is a method that considers the balance between related components of the VC. Using either a BMI(j) or BSA indexing (j) of the volume relationships provide slightly different measures. The first component, R1/R2 Rank, is an expression to avoid erroneously inflating the extreme values associated with the Abnormal Classification of Restrictive/Constrictive. The IF statement, (IIf(([Aj*5]>[E]*3],([Ej]/[Aj], [Aj/Ej])) measures the balance between the early filling volume and atrial contraction both indexed to j. These 2 values in the most balanced filling should represent nearly all of the filling volume. The Ej/Pj factors in the portion of the passive filling phase that is associated with the preferred E volume. The Aj/[Aj+Mj] determines what percentage of the non-early filling volume is contributed by the preferred A volume. Finally the 10 multiplier is simply to minimize the fractional component.

The VC groupings provide general buckets or categories of cardiac health or disease; however there are varying degrees of health or disease within those classifications. The VC_{MI} is a further sliding numeric scale within each class to indicate degree or severity. The VC_{MI} is a sliding scale measure of diastolic performance able to detect small changes within the VC classification model and secondarily to identify those at elevated risk of hospital admission. For example, it can determine if a patient is borderline in an Abnormal grouping and has less risk or significantly elevated risk within one of the classifications in the Abnormal grouping.

The same variability exists within the Normal grouping. In some embodiments, the VC_{MI} for classes in the Normal grouping is useful in evaluating cardiac performance such as with cardiac rehab exercise programs and medical therapies including proper medication titration for examples of medical applications. From a sporting perspective, the VC_{MI} for classes in the Normal grouping is useful for evaluating cardiac conditioning or developing training regimens based on an aerobic (diastolic/endurance conditioning) or anerobic (weight or strength training) focus in some embodiments. In some embodiments, the VC_{MI} for classes in the Normal grouping is useful for determining who can perform well for either a short duration (e.g., a sprinter), or maintain an acceptable level of performance for longer intervals.

VC provides a generalized classification. The VC_{MI} values generally correspond with relative level of risk within each classification. For example, for some indices, the VC_{MI} values may fall in a range of 0-200. Indexing to BMI will produce a different range than indexing to BMA. Within each abnormal classification, a lower VC_{MI} value corresponds to higher risk and a higher VC_{MI} value corresponds to lower risk. The lower the VC_{MI} number the higher the risk of admission within that classification. Between classifications, the VC_{MI} values can have similar ranges, especially in the Abnormal grouping. For example, when looking at the 30- and 90-day admissions a low range of VC_{MI} were seen in the Abnormal classifications of Mid Enhanced and Delayed Filling. These values could also be observed in the Early Enhanced classification; however, because the Early Enhanced classification is in the normal group, the risk of hospital admission for patients with these VC_{MI} values in the Early Enhanced classification is much lower than the risk for patients with classifications in the abnormal group and similar VC_{MI} values. Early Enhanced is a transitional class from Normal and can go nearly directly into any of the Abnormal Grouping classifications.

Using the VC_{MI} methods produced the best sustainable outcome combination with exclusion of computational “Outliers”. The Kaplan-Meier Curves of admissions following the INDEX echo for 30 and 90 days remained unchanged. (FIG. 19). The VC_{MI} improves the ability to assess small changes within VC classifications rather than requiring a change between classifications to determine improvement or demise in diastolic performance.

By first determining whether the patient or subject falls within the “Abnormal” grouping and then applying the VC_{MI}, a subset of the “Abnormal” grouping VC classification can be identified. The VC_{MI} provides a sliding scale within each of the classifications to further define the at-risk population for hospital admission. This is a benefit over current methods using a “bucket” approach of grouping similar characteristics as is used in both the Echo/Doppler current gold standard for diastolic function classification.

In some embodiments, determining a VC classification and a VCMI provides identifies a patient at elevated risk of admission to a hospital and provides additional information regarding a relative amount of elevated risk.

Some embodiments described herein provide devices, systems, and methods for identifying a heart failure patient at elevated risk of hospital readmission. Methods for identifying a heart failure patient at elevated risk of hospital readmission may employ any of the systems and/or devices described herein.

Finally, the terminology used herein is for describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments described herein were chosen and described in order to best explain the principles of the invention and the practical application, and to enable others of ordinary skill in the art to understand the invention for various embodiments with various modifications as are suited to the particular use contemplated.

### Abbreviations and Definitions

R1:R2 or R1/R2—the Early Passive Filling Rate (R1) divided by the Intermediate Passive Filling Rate (R2). The normal range is 4.00 to 16.00. Less than 4 represents decreased early left ventricular filling. Higher than 16 can either be due to enhanced atrial contribution or restrictive/constrictive pathology. Differentiation is accomplished using combinations of volume curve features.
A—Atrial contraction volume contribution
Ai—Atrial contraction indexed to BSA
Aj—Atrial contraction indexed to BMI
Ai %—Ai indexed to SVi
BMI—Body Mass Index
BSA—Body Surface Area
E—Early Filling Volume determined by the intersection of R2 with the VC curve
E*—An alternative to E using the intersection of R1 and R2 when E is poorly defined
Ei—Early Filling Volume indexed to body surface area
Ej—Early Filling Volume indexed to body mass index
Ei %—Ei indexed to FVi
FV—Filling Volume
FVi—FV indexed to BSA
HR—Heart Rate
LFV—Late Filling Volume is the measured volume following the intersection of R1 and R2.
LV—Left Ventricular
LVIT—Left ventricular inflow tract
M—Mid passive filling volume is the volume following E and terminating at A
MI—Moro Index
Mj—Mid passive filling volume indexed to body mass index
MVOi—Minute Ventricular Output BSA indexed
LA—Left atrium
LV—Left ventricle
RE—Rapid ejection
PrRE—Pre rapid ejection
PoRE—Post rapid ejection
R1—dP/dt of early ventricular filling phase
R2—dP/dt of mid ventricular filling phase
R3—dP/dt of atrial contraction ventricular filling phase
R4—dP/dt of rapid ejection during systolic phase
SV—Stroke Volume.
SVi—SV indexed to BSA.
VC—Volume Curve
VC_{MI}—Volume Curve-Moro Index

The invention claimed is:

1. A method for assessing cardiac function of a heart of a subject performed by a system or device including at least one processor, the method comprising:
receiving or accessing cardiac data obtained from imaging one or more complete cardiac cycles of the heart;
identifying a diastolic phase of the heart from the cardiac data;
identifying in the diastolic phase an early filling phase, an intermediate filling phase, and an atrial contraction phase from the cardiac data;
determining a left ventricular filling rate (R1) during the early filling phase from the cardiac data;
determining a left ventricular filling rate (R2) during the intermediate filling phase from the cardiac data;
determining a first left ventricular filling volume (E) during the early filling phase from the cardiac data;
determining a second left ventricular filling volume (A) during the atrial contraction phase from the cardiac data; and
generating a diastolic function classification based on at least R1, R2, E, and A.

2. (canceled)

3. The method of claim 1,
(i) wherein the diastolic function classification is based, at least in part, on at least a ratio of R1:R2; or
(ii) wherein the classification based on E is based on E as a percentage of the ventricular total filling volume (E %), as a percentage of a body surface area (Ei), or a combination of the two (Ei %); or
(iii) wherein the classification based on at least R1, R2, E, and A is based on A as a percentage of the ventricular total filling volume (A %), as a percentage of a body surface area (Ai), or a combination of the two (Ai %); or
(iv) one or more of (i), (ii), and (iii).

4.-7. (canceled)

8. The method of claim 1, wherein the classification is further based on a minute ventricular output (MVO) that is body surface area (BSA) indexed (MVOi).

9. The method of claim 1, further comprising calculating determining a left ventricular filling rate (R3) during the atrial contraction phase, wherein A is determined based on R3.

10. The method of claim 1, wherein the diastolic function classification includes one of normal, an early enhanced, a mid-enhanced, an atrial enhanced, an aerobic enhanced, a delayed filling, a restrictive/constrictive, or a hypovolemic classification.

11. The method of claim 1, wherein the diastolic function classification includes four classifications selected from the group consisting of: normal, an early enhanced, a mid-enhanced, an atrial enhanced, an aerobic enhanced, a delayed filling, a restrictive/constrictive, and a hypovolemic classification.

12. The method of claim 10,
(a) wherein the normal classification corresponds to an R1:R2 ratio of greater than about 4 and less than about 16, an E % of greater than about 50% and less than about 70%, and an A % of greater than about 25%; or
(b) wherein the early enhanced classification corresponds to an R1:R2 ratio of greater than about 4 and less than about 16, an E % of greater than about 50% and less than about 70%, and an A % of less than about 25%; or
(c) wherein the early enhanced classification corresponds to an R1:R2 ratio of less than about 16, and an E % of greater than about 70%; or
(d) wherein the mid-enhanced classification corresponds to an R1:R2 ratio of less than about 16, an E % of less than about 50%, and an A % of less than about 40%; or
(e) wherein the atrial enhanced classification corresponds to an R1:R2 ratio of less than about 16 and greater than about 4, an E % of less than about 50%, and an A % of greater than about 30%; or
(f) wherein the atrial enhanced classification corresponds to an R1:R2 ratio of greater than about 16, an A % of greater than about 30% and a minute ventricular output indexed by body surface area (MVOi) of less than about 2200; or
(g) wherein the aerobic enhanced classification corresponds to an R1:R2 ratio of greater than about 16, an A % of greater than about 30%, and a minute ventricular output indexed by body surface area (MVOi) of greater than about 2200; or
(h) wherein the delayed filling classification corresponds to an R1:R2 ratio of less than about 4, and an E % of greater than about 50% and less than about 70%; or
(i) wherein the restrictive/constrictive classification corresponds to an R1:R2 ratio of greater than about 16, and an A % of less than about 30%; or
(j) wherein the hypovolemic classification corresponds to an R1:R2 ratio of less than about 4, an E % of less than about 50% and an A % of greater than about 30%; or
(k) a combination more than one of (a), (b), (c), (d), (e), (f), (g), (h), (i), and (j).

13.-19. (canceled)

20. The method of claim 1,
further comprising the system or device displaying information regarding the generated diastolic function classification or a representation of the generated diastolic function classification on a display; or
further comprising transmitting information regarding the generated diastolic function classification to a health care provider or health care system of the subject.

21.-22. (canceled)

23. The method of claim 1, further comprising transmitting an alert to a health care provider or a health care system of the subject where the generated diastolic function classification is hypovolemic classification.

24. The method of claim 1, further comprising imaging the heart during one or more complete cardiac cycles to generate the cardiac data, wherein imaging the heart includes imaging using at least one of echocardiography, computed tomography (CT), and magnetic resonance imaging.

25. (canceled)

26. A system for automatic evaluation of cardiac function, comprising:
memory and at least one processor; and
a diastolic function classification module comprising instructions enabled upon execution in the memory of the host computing platform to:
receive or access cardiac data obtained from imaging one or more complete cardiac cycles of a heart;
identify a diastolic phase and a systolic phase of the heart based on the cardiac data;
identify in the diastolic phase an early filling phase, an intermediate filling phase, and an atrial contraction phase;
determine a left ventricular filling rate (R1) during the early filling phase from the cardiac data;
determine a left ventricular filling rate (R2) during the intermediate filling phase from the cardiac data;
determine a ventricular filling volume (E) during the early filling phase from the cardiac data;
determine a left ventricular filling volume (P) prior to the atrial contraction phase from the cardiac data;
determine a ventricular total filling volume (FV) from the cardiac data;
determine a left ventricular filling volume (A) during the atrial contraction phase from the cardiac data; and
generate a diastolic function classification based on R1, R2, E and A.

27. The system of claim 26,
(i) wherein the diastolic function classification is based, at least in part, on at least a ratio of R1:R2; or
(ii) wherein the classification based on E is based on E as a percentage of the ventricular total filling volume (E %), as a percentage of a body surface area (Ei), or a combination of the two (Ei %); or
(iii) wherein the classification based on at least R1, R2, E, and A is based on A as a percentage of the ventricular total filling volume (A %), as a percentage of a body surface area (Ai), or a combination of the two (Ai %); or
(iv) one or more of (i), (ii), and (iii).

28.-31. (canceled)

32. The system of claim 26, wherein the classification is further based on a minute ventricular output (MVO) that is body surface area (BSA) indexed (MVOi).

33. The system of claim 26,
wherein the instructions further include instructions to calculate determine a left ventricular filling rate (R3) during the atrial contraction phase, wherein A is determined based on R3.

34. The system of claim 26, wherein the diastolic function classification includes one of normal, an early enhanced, a mid-enhanced, an atrial enhanced, an aerobic enhanced, a delayed filling, a restrictive/constrictive or a hypovolemic classification.

35. The system of claim 26, wherein the diastolic function classification includes four classifications selected from the group consisting of: normal, an early enhanced, a mid-enhanced, an atrial enhanced, an aerobic enhanced, a delayed filling, a restrictive/constrictive, and a hypovolemic classification.

36. The system of claim 34,
(a) wherein the normal classification corresponds to an R1:R2 ratio of greater than about 4 and less than about 16, an E % of greater than about 50% and less than about 70%, and an A % of greater than about 25%; or
(b) wherein the early enhanced classification corresponds to an R1:R2 ratio of greater than about 4 and less than about 16, an E % of greater than about 50% and less than about 70%, and an A % of less than about 25%; or
(c) wherein the early enhanced classification corresponds to an R1:R2 ratio of less than about 16, and an E % of greater than about 70%; or
(d) wherein the mid-enhanced classification corresponds to an R1:R2 ratio of less than about 16, an E % of less than about 50%, and an A % of less than about 40%; or
(e) wherein the atrial enhanced classification corresponds to an R1:R2 ratio of less than about 16 and greater than about 4, an E % of less than about 50%, and an A % of greater than about 30%; or
(f) wherein the atrial enhanced classification corresponds to an R1:R2 ratio of greater than about 16, an A % of greater than about 30% and a minute ventricular output indexed by body surface area (MVOi) of less than about 2200; or
(g) wherein the aerobic enhanced classification corresponds to an R1:R2 ratio of greater than about 16, an A % of greater than about 30%, and a minute ventricular output indexed by body surface area (MVOi) of greater than about 2200; or
(h) wherein the delayed filling classification corresponds to an R1:R2 ratio of less than about 4, and an E % of greater than about 50% and less than about 70%; or
(i) wherein the restrictive/constrictive classification corresponds to an R1:R2 ratio of greater than about 16, and an A % of less than about 30%; or
(i) wherein the hypovolemic classification corresponds to an R1:R2 ratio of less than about 4, an E % of less than about 50% and an A % of greater than about 30%; or
(j) a combination more than one of (a), (b), (c), (d), (e), (f), (g), (h), (i), and (j).

37.-43. (canceled)

44. The system of claim 26, further comprising a display, wherein the instructions further include instructions to display information regarding the generated diastolic function classification or a representation of the generated diastolic function classification on the display.

45.-47. (canceled)

48. The system of claim 26, further comprising imaging instrumentation for imaging the heart, wherein the imaging instrumentation is configured for at least one of echocardiography, computed tomography (CT), and magnetic resonance imaging.

49.-52. (canceled)

53. A method for identifying a heart failure patient having an increased likelihood of hospital readmission, the method comprising:
performing the method of claim 1 to obtain a diastolic function classification of the patient's heart;
determining whether the patient falls in a normal group having a first likelihood of hospital readmission or in an abnormal group having a second likelihood of hospital readmission higher than the first likelihood based on the diastolic function classification; and
where the patient falls in the abnormal group, identifying the patient as having an increased likelihood of hospital readmission within a time period.

54. The method of claim 53, wherein the diastolic function classifications include normal, atrial enhanced, early enhanced, mid enhanced, delayed filling, restrictive/constrictive, and hypovolemic;
wherein the normal group includes the diastolic function classifications normal, atrial enhanced, and early enhanced; and
wherein the abnormal group includes the diastolic function classifications mid enhanced, delayed filling, restrictive/constrictive, and hypovolemic.

55. The method of claim 53, further comprising:
determining a Volume Curve Moro Index (VC_{MI}) from the cardiac data, the VC_{MI} corresponding to a likelihood of hospital readmission within the diastolic function classification, with a lower VC_{MI} value corresponding to a higher likelihood of hospital readmission within the specified time period and a higher lower VC_{MI} value corresponding to a lower likelihood of hospital readmission within the specified time period.

56.-60. (canceled)
